(19) United States
(12) Patent Application Publication
(10) Pub. No.: US 2024/0173230 A1
(43) Pub. Date: May 30, 2024

(54) FRAGRANCE COMPOSITION

(71) Applicant: GIVAUDAN SA, VERNIER (CH)

(72) Inventors: Stephen David WATKINS, Ashford Kent (GB); Andreas NATSCH, Uetikon (CH); Anne-Cecile KRALKA, Chanteloup-Les-Vignes (FR)

(21) Appl. No.: 18/549,200

(22) PCT Filed: Apr. 8, 2022
(86) PCT No.: PCT/EP2022/059365

§ 371 Date: Sep. 6, 2023

(30) Foreign Application Priority Data
Apr. 8, 2021 (GB) 2104969.7

(51) Int. Cl. A61K 8/37; A61K 8/31; A61K 8/33; A61K 8/34; A61K 8/35; A61K 8/49; A61K 8/58; A61K 8/9789; A61K 8/99; A61Q 15/00

(52) U.S. Cl. CPC A61K 8/37; A61K 8/31; A61K 8/33; A61K 8/34; A61K 8/342; A61K 8/35; A61K 8/4973; A61K 8/498; A61K 8/58; A61K 8/9789; A61K 8/99; A61Q 15/00; A61K 2800/77

(57) ABSTRACT

The present disclosure provides microbiome-friendly fragrance compositions adapted to mask, reduce or prevent malodours and consumer products comprising such a microbiome-friendly fragrance composition.

The present invention generally relates to a fragrance composition adapted to mask, reduce or prevent malodours. In particular, the present invention relates to a microbiome-friendly fragrance composition and to a consumer product comprising said microbiome-friendly fragrance composition.

It is well known that most body malodours are generated by the breakdown of secretions by bacteria present on the skin. For this reason, most commonly available deodorants or antiperspirants aim at killing odour-producing bacteria and usually contain antimicrobial agents and ethanol.

The antibacterial activity of a substance can be measured in a number of ways, but it is not un-common for the minimum inhibitory concentration (MIC) to be quoted for a particular anti-bacterial agent against a specific bacteria species.

In terms of skin microflora, the dominant microorganisms tend to be gram positive bacteria such as Staphylococcus, Corynebacterium and Propionibacterium, and so these are preferred examples of microorganisms to assess whether a substance is skin microbiome-friendly or not.

It is well documented that many fragrance ingredients, or perfumery raw materials (PRMs), have antimicrobial activity.

The MIC for a PRM may be obtained by adding increasing concentrations of PRM to a live culture of a single bacterial species and observing the effect. In an extreme case, the bacteria are killed (termed bactericidal), whereas in other cases the bacteria are not killed but sufficiently affected by the PRM, such that they are unable to multiply (termed bacteriostatic). At sufficiently low concentrations of PRMs, or with PRMs that have no/low antibacterial activity, the bacteria will continue to grow despite the presence of the PRM.

Nowadays, it is common practice to include antibacterial fragrance ingredients in deodorant or antiperspirant products, as they are expected to reduce bacterial activity and thereby also the formation of body malodours.

However, while some bacteria are involved in the formation of unpleasant body odours, others are highly beneficial for human skin health. In targeting the natural skin bacteria, there is a risk that pathogenic microorganisms may be allowed to colonise the skin.

It would therefore be highly desirable to be able to mask or prevent body malodours, while at the same time preserving the beneficial skin microbiome.

The present invention is based on the surprising discovery that specific combinations of fragrance ingredients provide efficient deodorancy without negatively affecting the skin microflora, especially the bacterial microflora in the underarm region.

Therefore, in a first aspect, the present invention provides a malodour counteracting fragrance composition, which can be used to mask or prevent body malodour.

In a second aspect, the present invention provides a consumer product comprising the malodour counteracting fragrance composition of the present invention.

In a third aspect, the present invention provides a method of masking, reducing and/or preventing body malodour, said method comprising the step of topically applying the malodour counteracting fragrance composition or the consumer of the present invention to the human body.

In the context of the present disclosure, the term “fragrance composition” relates to a mixture of fragrance ingredients, including if desired a suitable solvent or solvents and optionally also mixed with a solid substrate.

Fragrance ingredients are well known to those skilled in the art, and include those mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavor and Fragrance Materials—1991”, Allured Publishing Co. Wheaton, Ill. USA. Fragrance ingredients may include natural products, such as extracts, essential oils, absolutes, resinoids, resins, concretes, etc., and also synthetic substances such as hydrocarbons, alcohols, aldehydes, ketones, ethers, acids, esters, acetals, ketals, nitriles, etc., including saturated and unsaturated compounds, aliphatic, carbocyclic and heterocyclic compounds.

In the context of the present disclosure, the term “malodour counteracting fragrance composition” relates to a composition comprising one or more fragrance ingredients, which is able to mask, reduce or prevent the perception of a malodour.

The malodour counteracting fragrance composition of the present invention comprises one or more fragrance ingredients drawn from Group A and/or one or more fragrance ingredients drawn from Group B and/or one or more fragrance ingredients drawn from Group C. The malodour counteracting fragrance composition may optionally comprise additional fragrance ingredients, provided that the total concentration of fragrance ingredients selected from Group D does not exceed 10 wt % based on the total weight of the malodour counteracting fragrance composition.

The concentrations of said fragrance ingredients are selected such that the sum of the total concentration of fragrance ingredients drawn from Group A, multiplied by 5, plus the total concentration of fragrance ingredients drawn from Group B, multiplied by 4, plus the total concentration of fragrance ingredients drawn from Group C, multiplied by 3, is equal to or higher than 100. In other words:

5×[total concentration Group A]+4×[total concentration Group B]+3×[total concentration Group C]≥100;

The [total concentration Group A] is the sum of the individual concentrations of all fragrance ingredients drawn from Group A.

The [total concentration Group B] is the sum of the individual concentrations of all fragrance ingredients drawn from Group B.

The [total concentration Group C] is the sum of the individual concentrations of all fragrance ingredients drawn from Group C.

Throughout this disclosure, concentrations of fragrance ingredients are indicated in wt % based on the total weight of the malodour counteracting fragrance composition, if not indicated otherwise.

The Group A fragrance ingredients are selected from the group consisting of 3,8,8,11a-tetramethyldodecahydro-1H-3,5a-epoxynaphtho[2,1-c]oxepine (AMBERKETAL); (3aR,5aS,9aS,9bR)-3a,6,6,9a-tetramethyl-2,4,5,5a,7,8,9,9b-octahydro-1H-benzo[e][1]benzofuran (AMBROFIX, AMBROXAN); 3a,6,6,9a-tetramethyl-2,4,5,5a,7,8,9,9b-octahydro-1H-benzo[e][1]benzofuran (CETALOX); (E)-1-(2,6,6-trimethyl-1-cyclohexenyl)but-2-en-1-one (DAMASCONE BETA); 1-(2,6,6-trimethyl-1-cyclohex-3-enyl)but-2-en-1-one (DAMASCONE DELTA); 3-methyl-2-pentylcyclopent-2-enone (DIHYDRO JASMONE); 1-(1,2,8,8-tetramethyl-1,2,3,4,5,6,7,8-octahydronaphthalen-2-yl)ethanone (GEORGYWOOD); methyl 3-oxo-2-pentylcyclopentaneacetate (HEDIONE, CEPIONATE); benzyl 2-hydroxybenzoate (BENZYL SALICYLATE); (Z)-hex-3-en-1-yl 2-hydroxybenzoate (HEXENYL-3-CIS SALICYLATE); (E)-tridec-2-enenitrile (TRIDECENE-2-NITRILE); and mixtures thereof.

The Group B fragrance ingredients are selected from the group consisting of (E)-3-methyl-4-(2,6,6-trimethylcyclohex-2-en-1-yl)but-3-en-2-one (ISORALDEINE CETONE ALPHA, ALPHA ISOMETHYL IONONE); benzyl 3-phenylprop-2-enoate (BENZYL CINNAMATE); methyl 3-phenylprop-2-enoate (METHYL CINNAMATE); (E)-1-(2,6,6-trimethylcyclohex-2-en-1-yl)but-2-en-1-one (DAMASCONE ALPHA); 3-ethoxy-4-hydroxybenzaldehyde (ETHYL VANILLIN); 2-(phenoxy)ethyl 2-methylpropanoate (PHENOXY ETHYL ISOBUTYRATE); mixtures of isomers (E)- and (Z)-3-methyl-4-(2,6,6-trimethyl-2-cyclohexen-1-yl)-3-buten-2-one, and (E)-1-(2,6,6-trimethylcyclohex-2-en-1-yl)pent-1-en-3-one (ISORALDEINE 70, ISORALDEINE 95); 4-(4-hydroxyphenyl)butan-2-one (RASPBERRY KETONE); 2-(2-(4-methylcyclohex-3-en-1-yl)propyl)cyclopentan-1-one (NECTARYL); 2-cyclohexylidene-2-phenylacetonitrile (PEONILE); (3aR,6S,7aS)-3a,4,5,6,7,7a-hexahydro-1H-4,7-methanoinden-6-yl propanoate (FLOROCYCLENE); ethyl 2,6,6-trimethylcyclohexa-1,3-diene-1-carboxylate (ETHYL SAFRANATE); 1-((2E,5Z,9Z)-2,7,8-trimethylcyclododeca-2,5,9-trien-1-yl)ethanone (TRIMOFIX O); and mixtures thereof.

The Group C fragrance ingredients are selected from the group consisting of 2-(4-methyl-1-cyclohex-3-enyl)propan-2-yl acetate (TERPINYL ACETATE); (3aR,6S,7aS)-3a,4,5,6,7,7a-hexahydro-1H-4,7-methanoinden-6-yl acetate (JASMACYCLENE); 2-(tert-butyl)cyclohexyl acetate (AGRUMEX); (E)-3-phenylprop-2-en-1-ol (CINNAMIC ALCOHOL); (Z)-2-benzylideneheptanal (AMYL CINNAMIC ALDEHYDE); (E)-2-benzylideneoctanal (HEXYL CINNAMIC ALDEHYDE); (Z)-2-methyl-3-phenylacrylaldehyde (METHYL CINNAMIC ALDEHYDE); methyl 2-aminobenzoate (METHYL ANTHRANILATE); 4-phenylbutan-2-one (BENZYL ACETONE); (1 S,2R,5R)-2-ethoxy-2,6,6-trimethyl-9-methylenebicyclo[3.3.1]nonane (BOISIRIS); 3-methyl-5-propylcyclohex-2-enone (CELERY KETONE); 2-pentylcyclopentanone (QUINTONE); oxydibenzene (DIPHENYL OXIDE); tetrahydro-4-methyl-2-(2-methylpropyl)-2H-pyran-4-ol (FLOROSA); 1-(5,5-dimethylcyclohex-1-en-1-yl)pent-4-en-1-one (GALBANONE); 2-(2-hydroxypropan-2-yl)-5-methylcyclohexanol (GERANODYLE); 2-(8-isopropyl-6-methylbicyclo[2.2.2]oct-5-en-2-yl)-1,3-dioxolane (GLYCOLIERRAL); 3a-ethyl-6,6,9a-trimethyldodecahydronaphtho[1,2-c]furan (GRISALVA); benzo[d][1,3]dioxole-5-carbaldehyde (HELIOTROPINE CRYSTALS); (E)-4-(2,6,6-trimethylcyclohex-2-en-1-yl)but-3-en-2-one (IRISONE ALPHA); (2E,6Z)-3,7-dimethylnona-2,6-dienenitrile (LEMONILE); 4-methyl-1-propan-2-ylbicyclo[2.2.2]oct-2-ene-8-carboxylate (MAHAGONATE); (2-butan-2-yl-1-methylcyclohexyl) acetate (METAMBRATE); methyl 2-(methylamino)benzoate (DIMETHYL ANTHRANILATE); 1-((1S,8aS)-1,4,4,6-tetramethyl-2,3,3a,4,5,8-hexahydro-1H-5,8a-methanoazulen-7-yl)ethanone (METHYL CEDRYL KETONE); (Z)-3-methylcyclopentadec-5-enone (MUSCENONE); 2-ethoxynaphthalene (NEROLINE CRYSTALS); 2,4-dimethyl-2-(5,5,8,8-tetramethyl-5,6,7,8-tetrahydronaphthalen-2-yl)-1,3-dioxolane (OKOUMAL); 3,7-dimethyloctan-1-ol (PELARGOL); (E)-3,7-dimethylocta-2,6-dien-1-yl 2-phenylacetate (GERANYL PHENYL ACETATE); 2-phenylethyl 2-phenylacetate (PHENYL ETHYL PHENYL ACETATE); 5-pentyldihydrofuran-2(3H)-one (PRUNOLIDE); hexyl 2-hydroxybenzoate (HEXYL SALICYLATE); 1-(spiro[4.5]dec-7-en-7-yl)pent-4-en-1-one (SPIROGALBANONE); 3,7-dimethyloctan-3-ol (TETRAHYDRO LINALOOL); oxacyclohexadecan-2-one (THIBETOLIDE); 4-hydroxy-3-methoxybenzaldehyde (VANILLIN); 1-((1S,8aS)-1,4,4,6-tetramethyl-2,3,3a,4,5,8-hexahydro-1H-5,8a-methanoazulen-7-yl)ethanone (VERTOFIX COEUR); 2-methoxynaphtalene (YARA YARA); lemon oil, petitgrain oil, sage officinalis oil, and mixtures thereof.

The Group D fragrance ingredients are selected from the group consisting of mixtures of 3,7-dimethyloct-6-en-1-yl acetate and 3,7-dimethyloct-7-en-1-yl acetate (RHODINYL ACETATE); 4,8-dimethyl-2-propan-2-ylidene-3,3a,4,5,6,8a-hexahydro-1H-azulen-6-yl acetate (VETIVERYL ACETATE); (E)-2-methoxy-4-(prop-1-en-1-yl)phenyl acetate (ACETYL ISOEUGENOL); 2-phenylacetic acid (PHENYL ACETIC ACID); 2,6,10-trimethylundec-9-enal (ADOXAL); decan-1-ol (ALCOHOL C 10 DECYLIC); 10-UNDECEN-1-OL (ALCOHOL C 11 UNDECYLENIC); dodecan-1-01 (ALCOHOL C 12 LAURIC); tridecan-1-ol (ALCOHOL C 13 OXO); octan-1-ol (ALCOHOL C 8 OCTYLIC); nonan-1-ol (ALCOHOL C 9 NONYLIC); (4-isopropylphenyl)methanol (CUMINYL ALCOHOL); (4-prop-1-en-2-yl-1-cyclohexenyl)methanol (PERILLA ALCOHOL); decanal (ALDEHYDE C 10 DECYLIC); undecanal (ALDEHYDE C 110 UNDECYLIC); hexanal (ALDEHYDE C 6 HEXYLIC); heptanal (ALDEHYDE C 7 HEPTYLIC); nonanal (ALDEHYDE C 9 NONYLIC); (2E)-3-phenylprop-2-enal (CINNAMIC ALDEHYDE); 2-phenylpropanal (HYDRATROPIC ALDEHYDE); 3-phenylpropanal (PHENYL PROPIONIC ALDEHYDE); (2R,8aS)-1-[(E)-6-[(1 S)-2,2-dimethyl-6-methylidenecyclohexyl]-4-methylhex-3-enyl]-2,5,5,8a-tetramethyl-3,4,4a,6,7,8-hexahydro-1H-naphthalen-2-ol (AMBREINE); methyl 2-(benzylideneamino)benzoate (AMANDOLENE); (E)-methyl 2-((7-hydroxy-3,7-dimethyloctylidene)amino)benzoate (AURANTIOL); 6-isopropylquinoline (ISOPROPYL QUINOLINE); 2-(2-methylpropyl)quinoline (BUTYL QUINOLINE SECONDARY); (E)-3,7-dimethylocta-2,6-dien-1-yl butanoate (GERANYL BUTYRATE); (Z)-hex-3-en-1-yl butanoate (HEXENYL-3-CIS BUTYRATE); (4Z,8Z)-1,5,9-trimethyl-13-oxabicyclo[10.1.0]trideca-4,8-diene (CEDROXYDE); (E)-1-(2,6,6-trimethylcyclohex-2-en-1-yl)hepta-1,6-dien-3-one (CETONE V); (Z)-1,1-diethoxy-3,7-dimethylocta-2,6-diene (CITRATHAL R); 3,7-dimethyloct-6-en-1-ol (CITRONELLOL EXTRA); 3,7-dimethyloct-6-enenitrile (CITRONELLYL NITRILE); 2-((3,7-dimethyloct-6-en-1-yl)oxy)acetaldehyde (CITRONELLYL OXYACETALDEHYDE); 2-methoxy-4-methylphenol (CREOSOL); 2,4-dimethylcyclohex-3-ene-1-carbaldehyde (CYCLAL C); 3-(4-methylcyclohex-3-en-1-yl)butan-1-ol (CYCLOMETHYLENE CITRONELLOL); 8,8-dimethyl-1,2,3,4,5,6,7,8-octahydronaphthalene-2-carbaldehyde (CYCLOMYRAL); (E)-1-(2,6,6-trimethylcyclohexa-1,3-dien-1-yl)but-2-en-1-one (DAMASCENONE); 5-hexyloxolan-2-one (DECALACTONE GAMMA); 2-methoxy-4-propylphenol (DIHYDRO EUGENOL); 2-(4-methylcyclohexyl)propan-2-ol (DIHYDRO TERPINEOL); 2-(2-(3,3,5-trimethylcyclohexyl)acetyl)cyclopentanone (DIONE); 6-heptyltetrahydro-2H-pyran-2-one (DODECALACTONE DELTA); 5-octyloxolan-2-one (DODECALACTONE GAMMA); 4-allyl-2-methoxyphenol (EUGENOL); (2E,6Z)-3,7,11-trimethyldodeca-2,6,10-trien-1-ol (FARNESOL); 1-(3,5,5,6,8,8-hexamethyl-5,6,7,8-tetrahydronaphthalen-2-yl)ethanone (FIXOLIDE); 3-(4-ethylphenyl)-2,2-dimethylpropanal (FLORALOZONE); 4,4,8,8-tetramethyloctahydro-4a,7-methanonaphtho[1,8a-b]oxirene (FOLENOX); 4-isopropylcyclohexanol (FOLROSIA); 4,6,6,7,8,8-hexamethyl-1,3,4,6,7,8-hexahydrocyclopenta[g]isochromene (GALAXOLIDE); (E)-3,7-dimethylocta-2,6-dien-1-ol (GERANIOL); mixtures of thioterpenes (GRAPELIONE); methyl 7-isopropyl-1,4a-dimethyl-1,2,3,4,4a,4b,5,6,7,8,10,10a-dodecahydrophenanthrene-1-carboxylate (HERCOLYN); (Z)-hex-3-en-1-yl (Z)-hex-3-enoate (HEXENYL-3-CIS HEXENOATE); 7-hydroxy-3,7-dimethyloctanal (HYDROXYCITRONELLAL); 8,8-di(1H-indol-3-yl)-2,6-dimethyloctan-2-ol (INDOLENE); 6-butan-2-yl-quinoline (ISOBUTYL QUINOLINE-2); 3,7-dimethylocta-1,6-dien-3-yl 2-methylpropanoate (LINALYL ISOBUTYRATE); 2,4,6-trimethylcyclohex-3-enecarbaldehyde (ISOCYCLOCITRAL); (Z)-3-methyl-2-(pent-2-en-1-yl)cyclopent-2-enone (JASMONE CIS); (Z)-5-(hex-3-en-1-yl)-5-methyloxolan-2-one (JASMIN LACTONE GAMMA); 2,8,8-trimethyloctahydro-1H-4a,2-(epoxymethano)naphthalen-10-one (LACTOSCATONE); 8-isopropyl-1-oxaspiro[4.5]decan-2-one (LAITONE); (E)-3,7-dimethylocta-2,6-dienal (CITRAL); 3-hydroxy-2-methyl-4H-pyran-4-one (MALTOL); 4-[(3E)-4,8-dimethylnona-3,7-dienyl]pyridine (MARITIMA); 3-methyl-5-phenylpentanal (MEFRANAL); 5-hexyl-5-methyloxolan-2-one (METHYL DECALACTONE GAMMA); (E)-1,2-dimethoxy-4-(prop-1-en-1-yl)benzene (METHYL ISOEUGENOL); 2-methyldecanal (ALDEHYDE C 11 MOA); 4-(4-methylpent-3-en-1-yl)cyclohex-3-enecarbaldehyde (MYRALDENE); 3,7-dimethyl-2,6-octadien-1-ol (NEROL C); (Z)-3,7,11-trimethyldodeca-1,6,10-trien-3-ol (NEROLIDOL); 6,8-dimethylnonan-2-ol (NONADYL); 4,4a-dimethyl-6-(prop-1-en-2-yl)-4,4a,5,6,7,8-hexahydronaphthalen-2(3H)-one (NOOTKATONE CRYSTALS); (E)-3,7-dimethylocta-1,3,6-triene (OCIMENE); 3-methoxy-5-methylphenol (ORCINYL 3); p-cresol (CRESOL PARA); 6-methylquinoline (METHYL QUINOLINE PARA); 2-(6,6-dimethyl-5-bicyclo[2.2.1]heptanylidene)ethanol (PATCHOMINT); 2-phenoxyacetaldehyde (PHENOXY ACETALDEHYDE); 3-(6,6-dimethylbicyclo[3.1.1]hept-2-en-2-yl)propanal (PINOACETALDEHYDE); 3,7-dimethyloct-6-en-1-yl propanoate (CITRONELLYL PROPIONATE); 2-ethoxy-4-(isopropoxymethyl)phenol (PROPYL DIANTILIS); 6-(sec-butyl)quinoline (PYRALONE); mixtures of 3,7-dimethyloct-6-en-1-ol and 3,7-dimethyloct-7-en-1-ol (RHODINOL); 2-methyl-5-phenylpentan-1-ol (ROSAPHEN); 3-methyl-5-(2,2,3-trimethylcyclopent-3-en-1-yl)pentan-2-ol (SANDALORE); (3aR,4R,6S,7R,7aR)-6-methoxyoctahydro-1H-4,7-methanoindene-1-carbaldehyde (SCENTENAL); (E)-5-methylheptan-3-one oxime (STEMONE); (E)-6-ethyl-3-methyloct-6-en-1-ol (SUPER MUGUET); 1-methyl-4-(propan-2-ylidene)cyclohex-1-ene (TERPINOLENE); 3-(2,6,6-trimethyl-1-cyclohexen-1-yl)-prop-2-enenitrile (TERRANIL); 2,2,6-trimethylcyclohexyl-3-hexanol (TIMBEROL); 3-(benzo[d][1,3]dioxol-5-yl)-2-methylpropanal (TROPIONAL); 4-methyl-5-pentyldihydrofuran-2(3H)-one (METHYL TUBERATE); 6-hexyltetrahydro-2H-pyran-2-one (UNDECALACTONE DELTA); (Z)-cyclohexadec-5-enone (VELVIONE); 2-(tert-butyl)cyclohexanol (VERDOL); 2-ethyl-4-hydroxy-5-methylfuran-3(2H)-one (HOMOFURONOL); (Z)-non-6-en-1-ol (NONENOL-6-CIS); (2Z,6E)-2,6-nonadien-1-yl acetate (NONADIENYL ACETATE); (4-methylphenyl) acetate (PARA CRESYL ACETATE); (4S)-4,7,7-trimethyl-6-thiabicyclo[3.2.1]octane (CORPS PAMPLEMOUSSE); 2-(3-phenylpropyl)pyridine (CORPS RACINE VS); (2E,4E)-deca-2,4-dienal (DECADIENAL); (E)-dec-2-enal (DECENAL-2-TRANS); (E)-dodec-2-enal (DODECENAL); 8-methyl-1-oxaspiro[4.5]decan-2-one (METHYL LAITONE); (2E,6Z)-nona-2,6-dienal (LIME DIENES, NONADIENAL); (2Z,6E)-2,6-nonadien-1-ol (NONADIENOL-2,6); (Z)-non-6-enal (NONENAL-6-CIS); 4-ethyl-5-methylthiazol-2-ol (HYDROXY ETHYL METHYL THIAZOL); 2,6,6-trimethylcyclohexa-1,3-dienecarbaldehyde (SAFRANAL); 1-bicyclo [2.2.1]hept-5-en-2-yl-ethanone oxime (TERRAVERT); (E)-dodec-2-enal (ALDEHYDE MANDARINE); undec-10-enal (ALDEHYDE C 11 UNDECYLENIC); dodecanal (ALDEHYDE C 12 LAURIC); 2-methylundecanal (ALDEHYDE C 12 MNA); 3-(4-isopropylphenyl)-2-methylpropanal (CYCLAMEN ALDEHYDE); (E)-undec-9-enal (ALDEHYDE ISO C 11); 1-((2-(tert-butyl)cyclohexyl)oxy)butan-2-ol (AMBER CORE); 3-(4-(tert-butyl)phenyl)propanal (BOURGEONAL); 5-isopropyl-2-methylphenol (CARVACROL); 2H-chromen-2-one (COUMARIN CRYSTALS); 4-(4-hydroxy-4-methylpentyl)cyclohex-3-enecarbaldehyde (CYCLOHEXAL); (E)-dec-4-enal (DECENAL-4-TRANS); (E)-4-((3aS,7aS)-hexahydro-1H-4,7-methanoinden-5(6H)-ylidene)butanal (DUPICAL); (E)-3-methyl-5-(2,2,3-trimethylcyclopent-3-en-1-yl)pent-4-en-2-ol (EBANOL); 2-ethyl-3-hydroxy-4H-pyran-4-one (ETHYL MALTOL); methyl 2,4-dihydroxy-3,6-dimethylbenzoate (EVERNYL); 3-(4-methoxyphenyl)-2-methylpropanal (FENNALDEHYDE); 3a,6,6,9a-tetramethyldodecahydronaphtho[2,1-b]furan (FIXAMBRENE); 3-(3-isopropylphenyl)butanal (FLORHYDRAL); 2,4a,5,8a-tetramethyl-1,2,3,4,4a,7,8,8a-octahydronaphthalen-1-yl formate (OXYOCTALINE FORMATE); (E)-4-(2,6,6-trimethylcyclohex-1-en-1-yl)but-3-en-2-one (IONONE BETA); 4-formyl-2-methoxyphenyl 2-methylpropanoate (ISOBUTAVAN); (E)-2-methoxy-4-(prop-1-en-1-yl)phenol (ISOEUGENOL); (1-methyl-2-((1,2,2-trimethylbicyclo[3.1.0]hexan-3-yl)methyl)cyclopropyl)methanol (JAVANOL); 3-(4-(tert-butyl)phenyl)-2-methylpropanal (LILIAL); 2,2-dimethyl-3-(m-tolyl)propan-1-ol (MAJANTOL); (4-isopropylcyclohexyl)methanol (MAYOL); octahydro-2H-chromen-2-one (BICYCLO NONALACTONE); methyl non-2-ynoate (METHYL OCTYNE CARBONATE); 1-(2-naphtalenyl)-ethanone (ORANGER CRYSTALS); 4-(tert-butyl)cyclohexanol (BUTYL CYCLOHEXANOL PARA); 5-heptyldihydrofuran-2(3H)-one (PEACH PURE); 3-methyl-5-phenylpentan-1-ol (MEFROSOL); (E)-2-ethyl-4-(2,2,3-trimethylcyclopent-3-en-1-yl)but-2-en-1-ol (RADJANOL SUPER); dec-9-en-1-ol (ROSALVA); 3-((1R,2S,4R,6R)-5,5,6-trimethylbicyclo[2.2.1]heptan-2-yl)cyclohexanol (SANDELA); 2-(4-methylcyclohex-3-en-1-yl)propan-2-ol (TERPINEOL); 2-isopropyl-5-methylphenol (THYMOL CRYSTALS); 2,4-dimethylcyclohex-3-enecarbaldehyde (TRICYCLAL); almond oil sweet; amyris oil; bay oil; cedarwood oil; guaiacwood oil; sandalwood oil; cabreuva oil; cocoa absolute; cade oil; chamomile oil blue; cinnamon leaf oil; cassia oil; olibanum resinoid; fenugreek absolute; fucus oil; clove bud oil; clove leaf oil; orris resinoid; labdanum oil (DYNAMONE SB); labdanum resinoid; lavender absolute; lemongrass oil; lentisque resinoid; Litsea cubeba oil; nutmeg oil; spearmint oil; mousse de chene absolute; myrrh oil; narcisse absolute; opoponax oil; orange flower absolute; osmanthus absolute; palmarosa oil; pimento berry oil; styrax oil; tagete oil; thyme absolute white; thyme oil red; tonka beans absolute; tuberose absolute; vetiver oil; violet leaf absolute; origan oil; patchouli oil; and mixtures thereof.

Surprisingly, it has been found that by carefully selecting and combining certain fragrance ingredients according to the above specified rules, it is possible to provide a highly effective malodour counteracting fragrance composition that does not affect the skin microbiome.

In order to assess the effect on bacterial growth, individual fragrance ingredients were screened against three common skin bacteria (Staphylococcus, Corynebacterium and Propionibacterium), using standard methods known in the art. It was found that the fragrance ingredients of Groups A, B, and C were “microbiome-friendly”, i.e. that they only had a bacteriostatic MIC activity at a concentration higher than 0.1 wt %.

The malodour counteractancy was confirmed by sensory studies, an example of which is described below.

In particular, it was found that the fragrance ingredients of Group A displayed a very good malodour counteracting performance, those of Group B a good malodour counteracting performance, and those of Group C a moderate malodour counteracting performance.

The malodour counteracting fragrance composition of the present invention may comprise up to 10 wt % of fragrance ingredients drawn from Group D. Group D fragrance ingredients were found to affect microbial growth when used at higher concentrations and should therefore only be used in an amount such that the total concentration of these fragrance ingredients does not exceed 10 wt % based on the total weight of the malodour counteracting fragrance composition.

In an embodiment, the malodour counteracting fragrance composition of the present invention comprises at least one fragrance ingredient drawn from Group A, more preferably at least two, and most preferably at least three. Group A fragrance ingredients have been found to be particularly effective at counteracting malodour. In addition, fragrance compositions with a larger number of different fragrance ingredients tend to be perceived as more structured, more complex and overall more hedonically pleasing.

The following fragrance ingredients drawn from Group A have been found to be particularly well suited for the malodour counteracting fragrance composition of the present invention: 3,8,8,11a-tetramethyldodecahydro-1H-3,5a-epoxynaphtho[2,1-c]oxepine (AMBERKETAL); (3aR,5aS,9aS,9bR)-3a,6,6,9a-tetramethyl-2,4,5,5a,7,8,9,9b-octahydro-1H-benzo[e][1]benzofuran (AMBROFIX, AMBROXAN); benzyl 2-hydroxybenzoate (BENZYL SALICYLATE); 1-(2,6,6-trimethyl-1-cyclohex-3-enyl)but-2-en-1-one (DAMASCONE DELTA); methyl 3-oxo-2-pentylcyclopentaneacetate (HEDIONE, CEPIONATE); and (Z)-hex-3-en-1-yl 2-hydroxybenzoate (HEXENYL-3-CIS SALICYLATE). Thus, preferably, the malodour counteracting fragrance composition comprises one or more of these fragrance ingredients.

In an embodiment, the malodour counteracting fragrance composition of the present invention comprises more than one fragrance ingredients drawn from Group B and/or Group C, for instance at least two or at least three or at least four fragrance ingredients drawn from Group B and/or Group C.

The following fragrance ingredients drawn from Groups B and C have been found to be particularly well suited for the malodour counteracting fragrance composition of the present invention: 3-ethoxy-4-hydroxybenzaldehyde (ETHYL VANILLIN); (E)-2-benzylideneoctanal (HEXYL CINNAMIC ALDEHYDE); mixtures of isomers (E)- and (Z)-3-methyl-4-(2,6,6-trimethyl-2-cyclohexen-1-yl)-3-buten-2-one, and (E)-1-(2,6,6-trimethylcyclohex-2-en-1-yl)pent-1-en-3-one (ISORALDEINE 70, ISORALDEINE 95); lemon oil; 4-(4-hydroxyphenyl)butan-2-one (RASPBERRY KETONE); and of 2-(4-methyl-1-cyclohex-3-enyl)propan-2-yl acetate (TERPINYL ACETATE). Thus, preferably, the malodour counteracting fragrance composition comprises one or more of these fragrance ingredients.

In an embodiment, the malodour counteracting fragrance composition of the present invention comprises at least one fragrance ingredient drawn from Group A and at least two fragrance ingredients drawn from Group B and/or Group C; more preferably, it comprises at least one fragrance ingredient drawn from Group A and at least three fragrance ingredients drawn from Group B and/or Group C. For instance, the malodour counteracting fragrance composition may comprise at least one fragrance ingredient from Group A and at least two fragrance ingredients drawn from Group B; or at least one fragrance ingredient from Group A and at least two fragrance ingredients drawn from Group C; or at least one fragrance ingredient from Group A, at least one fragrance ingredient drawn from Group B and at least one fragrance ingredient from Group C. It has been found that, by combining at least one fragrance ingredient from Group A with several fragrance ingredients from Groups B and/or C, a fragrance composition may be obtained which not only has a strong malodour counteracting effect, but is also particularly pleasant from a hedonic point of view.

In an embodiment, the malodour counteracting fragrance composition of the present invention comprises at least five fragrance ingredients drawn from Group A and/or Group B and/or Group C, more preferably at least seven fragrance ingredients drawn from Group A and/or Group B and/or Group C, and most preferably at least eight fragrance ingredients drawn from Group A and/or Group B and/or Group C.

In an embodiment, the malodour counteracting fragrance composition of the present invention comprises 0.01 to 60 wt % of fragrance ingredients drawn from Group A. Preferably, the malodour counteracting fragrance composition comprises 0.05 to 40 wt % of fragrance ingredients drawn from Group A, more preferably 0.5 to 20 wt %, and most preferably 1 to 15 wt %.

In an embodiment, the malodour counteracting fragrance composition of the present invention comprises 0.05 to 60 wt % of fragrance ingredients drawn from Group B. Preferably, the malodour counteracting fragrance composition comprises 0.5 to 40 wt % of fragrance ingredients drawn from Group B, more preferably 0.5 to 20 wt %, and most preferably 1 to 15 wt %.

In an embodiment, the malodour counteracting fragrance composition of the present invention comprises 1 to 60 wt % of fragrance ingredients drawn from Group C. Preferably, the malodour counteracting fragrance composition comprises 2 to 30 wt % of fragrance ingredients drawn from Group C, more preferably 5 to 30 wt %, and most preferably 10 to 25 wt %.

It has been found that the above preferred concentration ranges provide a particularly effective malodour counteracting fragrance composition, especially if combined for two or even three groups.

Preferably, the malodour counteracting fragrance composition of the present invention comprises 1 to 15 wt % of fragrance ingredients drawn from Group A, and 10 to 40 wt % of fragrance ingredients drawn from Group B and/or Group C.

For example, the malodour counteracting fragrance composition may comprise 1 wt % of Group A fragrance ingredients, 10 wt % of Group B fragrance ingredients and 20 wt % of Group C fragrance ingredients. Alternatively, according to another example, the malodour counteracting fragrance composition may comprise 10 wt % of Group A fragrance ingredients and 20 wt % of Group C fragrance ingredients. Alternatively, according to yet another example, the malodour counteracting fragrance composition may comprise 15 wt % of Group A fragrance ingredients, 2 wt % of Group B fragrance ingredients and 7 wt % of Group C fragrance ingredients.

In an embodiment, the malodour counteracting fragrance composition of the present invention comprises less than 8 wt % of fragrance ingredients drawn from Group D, preferably less than 5 wt %, more preferably less than 3 wt %, and most preferably less than 1 wt %. It has been found that, by reducing the amount of Group D fragrance ingredients present, it is possible to make the malodour counteracting fragrance composition even more skin-friendly. This is particularly advantageous if the malodour counteracting fragrance composition is to be applied to sensitive skin or to sensitive skin areas, for instance in the underarm.

The efficacy of the malodour counteracting fragrance composition of the present invention may be further improved by including higher amounts of the fragrance ingredients of Groups A, B, and C.

Therefore, in an embodiment, the concentrations of said fragrance ingredients are selected such that

5×[total concentration Group A]+4×[total concentration Group B]+3×[total concentration Group C]≥110;

more preferably such that

5×[total concentration Group A]+4×[total concentration Group B]+3×[total concentration Group C]≥120;

even more preferably such that

5×[total concentration Group A]+4×[total concentration Group B]+3×[total concentration Group C]≥150;

and most preferably such that

5×[total concentration Group A]+4×[total concentration Group B]+3×[total concentration Group C]≥200;

In an embodiment, the concentrations of said fragrance ingredients are selected such that

4×[total concentration Group A]+4×[total concentration Group B]+3×[total concentration Group C]≥100;

more preferably such that

4×[total concentration Group A]+4×[total concentration Group B]+3×[total concentration Group C]≥110;

even more preferably such that

4×[total concentration Group A]+4×[total concentration Group B]+3×[total concentration Group C]≥120;

and most preferably such that

4×[total concentration Group A]+4×[total concentration Group B]+3×[total concentration Group C]≥150.

In an embodiment, the concentrations of said fragrance ingredients are selected such that

3×[total concentration Group A]+4×[total concentration Group B]+3×[total concentration Group C]≥100;

more preferably such that

3×[total concentration Group A]+4×[total concentration Group B]+3×[total concentration Group C]≥110;

even more preferably such that

3×[total concentration Group A]+2×[total concentration Group B]+1×[total concentration Group C]≥120; and

most preferably such that

3×[total concentration Group A]+2×[total concentration Group B]+1×[total concentration Group C]≥150.

The malodour counteracting fragrance composition of the present invention may optionally comprise one or more additional fragrance ingredients not belonging to either one of Groups A, B, C, and D. In particular, the malodour counteracting fragrance composition of the present invention may comprise one or more fragrance ingredients selected from the group consisting of 4-methoxybenzyl acetate (ANISYL ACETATE); benzyl acetate (BENZYL ACETATE); 2-methyl-6-methyleneoct-7-en-2-yl acetate (BERGAMYL ACETATE); (2S,4S)-1,7,7-trimethylbicyclo[2.2.1]heptan-2-yl acetate (BORNYL ACETATE LAEVO); butyl acetate (BUTYL ACETATE); ((3R,3aS,7R,8aS)-3,8,8-trimethyl-2,3,4,7,8,8a-hexahydro-1H-3a,7-methanoazulen-6-yl)methyl acetate (CEDRENYL ACETATE); (1S,6R,8aR)-1,4,4,6-tetramethyloctahydro-1H-5,8a-methanoazulen-6-yl acetate (CEDRYL ACETATE); (E)-3-phenylprop-2-en-1-yl acetate (CINNAMYL ACETATE); 3,7-dimethyloct-6-en-1-yl acetate (CITRONELLYL ACETATE); 2-cyclohexylethyl acetate (CYCLOHEXYL ETHYL ACETATE); 1,2,3,4,4a,5,6,7,8,8a-decahydronaphthalen-2-yl acetate (DECAHYDRO NAPHTYL ACETATE BETA); 2-methyl-1-phenylpropan-2-yl acetate (DIMETHYL BENZYL CARBINYL ACETATE); 2-methyl-4-phenylbutan-2-yl acetate (DIMETHYL PHENYL ETHYL CARBINYL ACETATE); (2S)-1,3,3-trimethylbicyclo[2.2.1]heptan-2-yl acetate (FENCHYL ACETATE); 2-(3,8-dimethyl-1,2,3,4,5,6,7,8-octahydroazulen-5-yl)propan-2-yl acetate (GUAIYL ACETATE); (E)-3,7-dimethylocta-2,6-dien-1-yl acetate (GERANYL ACETATE); (E/Z)-hex-3-en-1-yl acetate (HEXENYL ACETATE CIS & TRANS); hexyl acetate (HEXYL ACETATE); 3-methylbutyl acetate (ISOAMYL ACETATE); (2-methoxy-4-prop-1-enylphenyl) acetate (ISOCARYOL ACETATE); 3,7-dimethylocta-1,6-dien-3-yl acetate (LINALYL ACETATE); (Z)-3,7-dimethylnona-1,6-dien-3-yl acetate (ETHYL LINALYL ACETATE); 2-(4-methylcyclohexyl)propan-2-yl acetate (MENTHANYL ACETATE); (4-(4-methylpent-3-en-1-yl)cyclohex-3-en-1-yl)methyl acetate (MYRALDYL ACETATE); (Z)-3,7-dimethylocta-2,6-dien-1-yl acetate (NERYL ACETATE); 3,5,5-trimethylhexyl acetate (ISONONANYL ACETATE); 2-(6,6-dimethylbicyclo[3.1.1]hept-2-en-2-yl)ethyl acetate (NOPYL ACETATE); oct-1-en-3-yl acetate (OCTENYL ACETATE); 4-(tert-butyl)cyclohexyl acetate (BUTYL CYCLOHEXYL ACETATE PARA); allyl 2-phenoxyacetate (ACETATE PA); 2-phenylethyl acetate (PHENYL ETHYL ACETATE); 3-phenylpropyl acetate (PHENYL PROPYL ACETATE); 3-methylbut-2-en-1-yl acetate (PRENYL ACETATE); 3,7-dimethyloctan-3-yl acetate (TETRAHYDRO LINALYL ACETATE); (2-benzyl-1,3-dioxolan-4-yl)methanol (ACETAL CD); (2-(1-ethoxyethoxy)ethyl)benzene (ACETAL E); (2-(1-propoxyethoxy)ethyl)benzene (ACETAL R); 1-(4-methoxyphenyl)ethanone (ACETANISOLE); ethyl 3-oxobutanoate (ETHYL ACETOACETATE); 1-phenylethanone (ACETOPHENONE); (E)-methyl 2-(((2,4-dimethylcyclohex-3-en-1-yl)methylene)amino)benzoate (AGRUMEA); (4-methoxyphenyl)methanol (ANISYL ALCOHOL); phenylmethanol (BENZYL ALCOHOL); hexan-1-ol (ALCOHOL C 6 HEXYLIC); (1S,2R,4R)-1,3,3-trimethylbicyclo[2.2.1]heptan-2-ol (FENCHYL ALCOHOL); isopentanol (AMYL ALCOHOL ISO); 2-phenoxyethanol (PHENOXY ETHYL ALCOHOL); 2-phenylethanol (PHENYL ETHYL ALCOHOL); 3-phenylpropan-1-ol (PHENYL PROPYL ALCOHOL); octanal (ALDEHYDE C 8 OCTYLIC); 3,5,5-trimethylhexanal (ALDEHYDE C 9 ISONONYLIC); 4-isopropylbenzaldehyde (CUMINIC ALDEHYDE); 4-methylbenzaldehyde (TOLYL ALDEHYDE PARA); heptan-2-one (HEPTONE); prop-2-enyl 2-(3-methylbutoxy)acetate (ALLYL AMYL GLYCOLATE); 2,5,5-trimethyl-1,2,3,4,4a,5,6,7-octahydronaphthalen-2-ol (mixture of isomers) (AMBRINOL); 1-octene-3-ol (AMYL VINYL CARBINOL); (E)-methyl octa-4,7-dienoate (ANAPEAR); phenylethyl 2-methylbutanoate (ANATOLYL); (E)-1-methoxy-4-(prop-1-en-1-yl)benzene (ANETHOLE); (2-(isopentyloxy)ethyl)benzene (ANTHER); [(Z)-hex-3-enyl]2-aminobenzoate (HEXENYL-3-CIS ANTHRANILATE); 2-phenylethyl 2-aminobenzoate (PHENYL ETHYL ANTHRANILATE); 1-(3,3-dimethylcyclohexyl)ethyl formate (APHERMATE); 4-methoxybenzaldehyde (AUBEPINE PARA CRESOL); benzaldehyde (BENZALDEHYDE); pentyl benzoate (AMYL BENZOATE); benzyl benzoate (BENZYL BENZOATE); ethyl benzoate (ETHYL BENZOATE); (Z)-hex-3-en-1-yl benzoate (HEXENYL-3-CIS BENZOATE); hexyl benzoate (HEXYL BENZOATE); 2-methylpropyl benzoate (ISOBUTYL BENZOATE); 3,7-dimethylocta-1,6-dien-3-yl benzoate (LINALYL BENZOATE); methyl benzoate (METHYL BENZOATE); 2-phenylethyl benzoate (PHENYL ETHYL BENZOATE); diphenylmethanone (BENZOPHENONE); (methoxymethyl)benzene (BENZYL METHYL ETHER); ethyl 6-acetoxyhexanoate (BERRYFLOR); (Z)-hex-3-en-1-yl 2-methyl butanoate (HEXENYL-3-CIS METHYL-2-BUTYRATE); (E)-1-methyl-4-(6-methylhepta-2,5-dien-2-yl)cyclohex-1-ene (BISABOLENE); (ethoxymethoxy)cyclododecane (BOISAMBRENE FORTE); (1S,2S,4S)-1,7,7-trimethylbicyclo[2.2.1]heptan-2-ol (BORNEOL CRYSTALS); 2,6-di-tert-butyl-4-methylphenol (BUTYL HYDROXY TOLUENE); pentyl butanoate (AMYL BUTYRATE); benzyl butanoate (BENZYL BUTYRATE); 3,7-dimethyloct-6-en-1-yl butanoate (CITRONELLYL BUTYRATE); 2-methyl-1-phenylpropan-2-yl butanoate (DIMETHYL BENZYL CARBINYL BUTYRATE); ethyl butanoate (ETHYL BUTYRATE); hexyl butanoate (HEXYL BUTYRATE); 3,7-dimethylocta-1,6-dien-3-yl butanoate (LINALYL BUTYRATE); 2-phenylethyl butanoate (PHENYL ETHYL BUTYRATE); 1-butoxy-1-oxopropan-2-yl butanoate (BUTYL BUTYRO LACTATE); 1,2,4-trimethoxy-5-propylbenzene (CALMODE); 8-methyl-1,5-benzodioxepin-3-one (CALONE 1951); (1S,4R)-2,2-dimethyl-3-methylenebicyclo[2.2.1]heptane (CAMPHENE); (1S,4S)-1,7,7-trimethylbicyclo[2.2.1]heptan-2-one (CAMPHOR); prop-2-enyl hexanoate (ALLYL CAPROATE); pentyl hexanoare (AMYL CAPROATE); ethyl hexanoate (ETHYL HEXANOATE); ethyl octanoate (ETHYL OCTANOATE); (4-methylphenyl) octanoate (CRESYL CAPRYLATE PARA); diethylene glycol mono methyl ether (CARBITOL); (5R)-2-methyl-5-prop-1-en-2-ylcyclohex-2-en-1-one (CARVONE LAEVO); (4Z)-4,11,11-trimethyl-8-methylenebicyclo(7.2.0)undec-4-ene (CARYOPHYLLENE); 1,1,2,3,3-pentamethyl-2,3,6,7-tetrahydro-1H-inden-4(5H)-one (CASHMERAN); (1S,6R,8aR)-1,4,4,6-tetramethyloctahydro-1H-5,8a-methanoazulen-6-ol (CEDROL CRYSTALS); (1R,6S,8aS)-6-methoxy-1,4,4,6-tetramethyloctahydro-1H-5,8a-methanoazulene (CEDRYL METHYL ETHER); 2,2-dimethyl-2-pheylethyl propanoate (PIVAROSE); 2-methyl-4-(2,6,6-trimethylcyclohex-2-en-1-yl)butanal (CETONAL); (E)-3-phenylprop-2-enenitrile (CINNAMALVA); 3-phenylprop-2-enyl 3-phenylprop-2-enoate (CINNAMYL CINNAMATE); ethyl 3-phenylprop-2-enoate (ETHYL CINNAMATE); 3,7-dimethylocta-1,6-dien-3-yl 3-phenylprop-2-enoate (LINALYL CINNAMATE); 2-phenylethyl 3-phenylprop-2-enoate (PHENYL ETHYL CINNAMATE); 3,7-dimethyloct-6-enal (CITRONELLAL); (Z)-cycloheptadec-9-enone (CIVETTONE); dodecanenitrile (CLONAL); 2-(tert-pentyl)cyclohexyl acetate (CONIFERAN); 2-hydroxy-3-methylcyclopent-2-enone (CORYLONE); (E)-3,7-dimethylocta-2,6-dien-1-yl but-2-enoate (GERANYL CROTONATE); 4-isopropylbenzonitrile (CUMIN NITRILE); allyl 2-(cyclohexyloxy)acetate (CYCLOGALBANATE); prop-2-enyl 3-cyclohexylpropanoate (ALLYL CYCLOHEXYL PROPIONATE); methyl 2-cyclopentylideneacetate (CYCLOPIDENE); hexyl 2-methylbutanoate (CYDRANE); 3-tert-butyl-cyclohexyl acetate (CYPERATE); Benzoic acid, 2-[[2-methyl-3-[4-(1-methylethyl)phenyl]propylidene]amino]-, methyl ester (CYRANTIOL, CYCLAMENTIOL); (2E,4Z)-ethyl deca-2,4-dienoate (ETHYL DECADIENOATE); 6-pentyltetrahydro-2H-pyran-2-one (DECALACTONE DELTA); decanenitrile (DECANONITRILE); 6-isopropyloctahydronaphthalen-2(1H)-one (DECATONE); 1-methoxydecane (DECYL METHYL ETHER); (1 S,6S)-3,7,7-trimethylbicyclo[4.1.0]hept-3-ene (DELTA-3 CARENE); 2-(sec-butyl)-1-vinylcyclohexyl acetate (DIHYDRO AMBRATE); 1-methoxy-4-propylbenzene (DIHYDRO ANETHOLE); (3aa,4a,5a,7a,7aa)-octahydro-4,7-methano-1H-inden-5-yl acetate (DIHYDRO CYCLACET); (Z)-3,7,11-trimethyldodeca-6,10-dienal (DIHYDRO FARNESAL); 4-(2,6,6-trimethylcyclohex-1-en-1-yl)butan-2-one (DIHYDRO IONONE BETA); methyl 2-hexyl-3-oxocyclopentane-1-carboxylate (METHYL DIHYDRO ISOJASMONATE); 3,7-dimethyloct-6-en-3-ol (DIHYDRO LINALOOL); 2,6-dimethyloct-7-en-2-ol (DIHYDRO MYRCENOL); (Z)-2-benzylideneheptanal dimethylacetal (AMYL CINNAMIC ALDEHYDE DIMETHYL ACETAL); (1,1-dimethoxypropan-2-yl)benzene (HYDRATROPIC ALDEHYDE DIMETHYL ACETAL); (E)-1,1-dimethoxy-3,7-dimethylocta-2,6-diene (CITRAL DIMETHYL ACETAL); 8,8-dimethoxy-2,6-dimethyloctan-2-ol (HYDROXYCITRONELLAL DIMETHYL ACETAL); 2-methyl-1-phenylpropan-2-ol (DIMETHYL BENZYL CARBINOL); 1,4-dimethoxybenzene (DIMETHYL HYDROQUINONE CRYSTALS); 4,7-dimethyloct-6-en-3-one (DIMETHYL OCTENONE); 2-methyl-4-phenylbutan-2-ol (DIMETHYL PHENYL ETHYL CARBINOL); 2,6-dimethylheptan-2-ol (DIMETOL); 2,6-dimethyloct-7-en-2-ol (DIMYRCETOL); 1-methoxyhexane (DIOLA); 2-heptan-3-yl-1,3-dioxolane (DIOLANE); diphenylmethane (DIPHENYL METHANE); (1 aS,2aR,3R, 5aS,7R,7aR)-octahydro-3,6,6,7a-tetramethyl-2H-2a,7-methanozuleno 5,6-b oxirene (CEDRENE EPOXIDE); 1-allyl-4-methoxybenzene (ESTRAGOLE);RUM ETHER (CAS #8030-89-5); 3,7-dimethyloct-6-en-1-yl ethyl oxalate (CITRONELLYL ETHOXALATE); ethyl 2-ethylhexanoate (ETHYL ETHYL CAPROATE); 6-methylheptan-3-one (ETHYL ISOAMYL KETONE); (E)-3,7-dimethylnona-1,6-dien-3-ol (ETHYL LINALOOL); octan-3-one (ETHYL AMYL KETONE); (3E,6E)-3,7,11-trimethyldodeca-1,3,6,10-tetraene (FARNESENE); (1R,4S)-1,3,3-trimethylbicyclo[2.2.1]heptan-2-one (FENCHONE LAEVO); 5-(allyloxy)-3a,4,5,6,7,7a-hexahydro-1H-4,7-methanoindene (FLEUROXENE); 2,6-dimethyloct-7-en-2-ol (FLORALYM); 2-(tert-butyl)cyclohexyl ethyl carbonate (FLORAMAT); 2,4,6-trimethyl-4-phenyl-1,3-dioxane (FLOROPAL); methyl oct-2-ynoate (FOLIONE); benzyl formate (BENZYL FORMATE); 3,7-dimethyloct-6-en-1-yl formate (CITRONELLYL FORMATE); 1,2,3,4,4a,5,6,7,8,8a-decahydronaphthalen-2-yl formate (DECAHYDRO NAPHTYL FORMATE BETA); (E)-3,7-dimethylocta-2,6-dien-1-yl formate (GERANYL FORMATE); (Z)-hex-3-en-1-yl formate (HEXENYL-3-CIS FORMATE); 3,7-dimethylocta-1,6-dien-3-yl formate (LINALYL FORMATE); 2-phenylethyl formate (PHENYL ETHYL FORMATE); ethyl methyl phenyl glycidate (STRAWBERRY PURE); ethyl 2-(2,4-dimethyl-1,3-dioxolan-2-yl)acetate (FRAISTONE); 2-(sec-butyl)cyclohexanone (FRESKOMENTHE); ethyl 2-(2-methyl-1,3-dioxolan-2-yl)acetate (FRUCTONE); (3aS,4S,7R,7aS)-ethyl octahydro-1H-4,7-methanoindene-3a-carboxylate (FRUITATE); dihexyl-but-2-enedioate (DIHEXYL FUMARATE); 2-methoxyphenol (GUAIACOL); 1-phenylethyl acetate (GARDENOL); (3aR,6S,7aS)-3a,4,5,6,7,7a-hexahydro-1H-4,7-methanoinden-6-yl 2-methyl propanoate (GARDOCYCLENE); 3,7-dimethyl-octa-2,6-dienenitrile (GERANITRILE); (E)-6,10-dimethylundeca-5,9-dien-2-one (GERANYL ACETONE); ethyl 2-ethyl-6,6-dimethylcyclohex-2-enecarboxylate (GIVESCONE); 2-butyl-4,6-dimethyl-3,6-dihydro-2H-pyran (GYRANE); (3R,5R)-3-ethoxy-1,1,5-trimethylcyclohexane (HERBAVERT); 2-butyl-4,4,6-trimethyl-1,3-dioxane (HERBOXANE); hexane-2,4-diol (HEXANEDIOL-2,4); 1-(4-methoxy-2,2,6,6-tetramethylcyclohex-3-en-1-yl)ethanone (KEFARENE); (Z)-hex-3-en-1-ol (HEXENOL-3-CIS); 4,4a,5,9b-tetrahydroindeno[1,2-d][1,3]dioxine (INDOFLOR); 4-methyl-2-(2-methylprop-1-enyl)-3,6-dihydro-2H-pyran (NEROL OXIDE); (E)-1-(2,6,6-trimethylcyclohex-2-en-1-yl)pent-1-en-3-one (IRALIA PURE); (E)-3-methyl-4-(2,6,6-trimethylcyclohex-2-en-1-yl)but-3-en-2-one (IRISANTHEME); (E)-4-(2,6,6-trimethylcyclohex-2-en-1-yl)but-3-en-2-one (IRISONE PURE); (E)-4-(2,5,6,6-tetramethyl-1-cyclohex-2-enyl)but-3-en-2-one (IRONAL); (E)-4-(2,5,6,6-tetramethylcyclohex-2-en-1-yl)but-3-en-2-one (IRONE ALPHA, IRONE E); (E)-4-(2,5,6,6-tetramethylcyclohex-2-en-1-yl)but-3-en-2-one (IRONE F); 1-(2,3,8,8-tetramethyl-1,2,3,4,5,6,7,8-octahydronaphthalen-2-yl)ethanone (ISO E SUPER); diethyl propanedioate (DIETHYL MALONATE); benzyl 2-methylpropanoate (BENZYL ISOBUTYRATE); (4-methylphenyl) 2-methylpropanoate (CRESYL ISOBUTYRATE PARA); ethyl 2-methylpropanoate (ETHYL ISOBUTYRATE); (E)-3,7-dimethylocta-2,6-dien-1-yl 2-methylpropanoate (GERANYL ISOBUTYRATE); (Z)-hex-3-en-1-yl 2-methylpropanoate (HEXENYL-3-CIS ISOBUTYRATE); hexyl 2-methylpropanoate (HEXYL ISOBUTYRATE); 2-methylpropyl 2-methylpropanoate (ISOBUTYL ISOBUTYRATE); 2-methyl-4-oxo-4H-pyran-3-yl 2-methylpropanoate (MALTYL ISOBUTYRATE); 3,7-dimethyloct-6-en-1-yl 2-methylpropanoate (CITRONELLYL ISOBUTYRATE); 2-phenylethyl 2-methylpropanoate (PHENYL ETHYL ISOBUTYRATE); 2-hexylcyclopent-2-en-1-one (ISOJASMONE B 11); 2-isopropyl-5-methylcyclohexanone (ISOMENTHONE DL); 4-methylpent-4-en-2-yl 2-methylpropanoate (ISOPENTYRATE); 5-methyl-2-(prop-1-en-2-yl)cyclohexanol (ISOPULEGOL); benzyl 3-methylbutanoate (BENZYL ISOVALERATE); 2-phenylethyl 3-methylbutanoate (PHENYL ETHYL ISOVALERATE); 3-pentyltetrahydro-2H-pyran-4-yl acetate (JASMOPYRANE); (E)-6-(pent-3-en-1-yl)tetrahydro-2H-pyran-2-one (JASMOLACTONE); 3-butyl-5-methyltetrahydro-2H-pyran-4-yl acetate (JASMONYL); 4-(1-ethoxyethenyl)-3,3,5,5-tetramethylcyclohexan-1-one (KEPHALIS); (Z)-3,4,5,6,6-pentamethylhept-3-en-2-one (KOAVONE); 3,4,5,6,6-pentamethylheptan-2-ol (KOHINOOL); (E)-2,4,4,7-tetramethylnona-6,8-dien-3-one (LABIENONE); (Z)-6-(pent-2-en-1-yl)tetrahydro-2H-pyran-2-one (JASMIN LACTONE DELTA); (Z)-1-(1-ethoxyethoxy)hex-3-ene (LEAF ACETAL); bicyclo[2.2.2]oct-5-ene-2-carboxaldehyde (MACEAL); (Z)-hex-3-en-1-yl methyl carbonate (LIFFAROME); 2,2,6-trimethyl-6-vinyltetrahydro-2H-pyran (LIMETOL); 3,7-dimethylocta-1,6-dien-3-ol (LINALOOL); 2-(4-methylcyclohex-3-en-1-yl)propan-2-ol (LINDENOL); ethyl 2-methylpentanoate (MANZANATE); 2,6-dimethylhept-5-enal (MELONAL); 2-isopropyl-5-methylcyclohexanone (MENTHONE); 2-isopropyl-5-methylcyclohexanol (MENTHOL-DL); 4-(4-methoxyphenyl)butan-2-one (METHOXY PHENYL BUTANONE); 6-methoxy-2,6-dimethylheptanal (METHOXYMELONAL); 1-(p-tolyl)ethanone (METHYL ACETOPHENONE); heptan-2-one (METHYL AMYL KETONE); butyl 2-methylpentanoate (METHYL CAMOMILLE); 2-ethoxy-4-(methoxymethyl)phenol (METHYL DIANTILIS); 2-methoxy-1,1′-biphenyl (METHYL DIPHENYL ETHER); 6-methylhept-5-en-2-one (METHYL HEPTENONE); octan-2-one (METHYL HEXYL KETONE); (Z)-2-methyl-4-(2,6,6-trimethyl-1-cyclohex-2-enyl)but-2-enal (METHYL IONAL BETA); 1-(2,6,6-trimethyl-1-cyclohex-2-enyl)pent-1-en-3-one (ALPHA METHYL IONONE); 5-butyl-4-methyloxolan-2-one (METHYL OCTALACTONE); 6,6-dimethoxy-2,5,5-trimethylhex-2-ene (METHYL PAMPLEMOUSSE); undecan-2-one (METHYL NONYL KETONE); isopropyl 2-methylbutanoate (ISOPROPYL METHYL-2-BUTYRATE); (E)-3-methyl-4-(2,6,6-trimethylcyclohex-1-en-1-yl)but-3-en-2-one (METHYLIONANTHEME); 1a,3,3,4,6,6-hexamethyl-1a,2,3,4,5,6,7,7a-octahydronaphtho[2,3-b]oxirene (MOXALONE); 3-methylcyclopentadecanone (MUSCONE); 4′-tert-butyl-2′,6′-dimethyl-3′,5′-dinitroacetophenone (MUSK KETONE); 1-(4-tert-butyl-2,6-dimethyl-3,5-dinitrophenyl)ethanone 0; 7-methyl-3-methyleneocta-1,6-diene (MYRCENE 90); (E)-methyl non-2-enoate (NEOFOLIONE); 6-propyltetrahydro-2H-pyran-2-one (OCTALACTONE DELTA); 5-butyloxolan-2-one (OCTALACTONE GAMMA); ethyl oct-2-enoate (ETHYL OCTENOATE-2); 4-(2-methoxypropan-2-yl)-1-methylcyclohexene (ORANGE FLOWER ETHER); tetradecanenitrile (ORANILE); 4-(tert-pentyl)cyclohexanone (ORIVONE); 7-methoxy-3,7-dimethyloctan-2-ol (OSYROL); mixture of terpene oxides (LIME OXIDE); 2-(5-methyl-5-vinyltetrahydrofuran-2-yl)propan-2-ol (LINALOOL OXIDE); methyl 2-hydroxy-5-methylbenzoate (METHYL CRESOTATE PARA); 1-methoxy-4-methylbenzene (CRESYL METHYL ETHER PARA); 1-methyl-4-propan-2-ylbenzene (CYMENE PARA); 4-(tert-butyl)cyclohexanone (BUTYL CYCLOHEXANONE PARA TERT); (2-methoxyethyl)benzene (PANDANOL); 1,1-dimethoxynon-2-yne (PARMAVERT); ethyl nonanoate (ETHYL PELARGONATE); 2-pentylcyclopent-2-en-1-one (PENTYL CYCLOPENTENONE); 2-methylpentyl 2-methylpentanoate (PERANAT); 2-(phenoxy)eth-2-yl acetate (ETHYL PHENOXY ACETATE); pentyl 2-phenylacetate (AMYL PHENYL ACETATE); benzyl 2-phenylacetate (BENZYL PHENYL ACETATE); ethyl 2-phenylacetate (ETHYL PHENYL ACETATE); 2-methoxy-4-(2-propenyl)phenyl 2-phenyl acetate (EUGENYL PHENYL ACETATE); 2-methylpropyl 2-phenylacetate (ISOBUTYL PHENYL ACETATE); 3,7-dimethylocta-1,6-dien-3-yl 2-phenyl-acetate (LINALYL PHENYL ACETATE); methyl 2-phenylacetate (METHYL PHENYL ACETATE); (4-methylphenyl) 2-phenylacetate (CRESYL PHENYL ACETATE PARA); ethyl 3-phenyloxirane-2-carboxylate (ETHYL PHENYL GLYCIDATE); 2,2,7,7-tetramethyltricyclo[6.2.1.01,6]undecan-5-one (ISOLONGIFOLANONE, PICONIA); 2,6,6-trimethylbicyclo[3.1.1]hept-2-ene (PINENE ALPHA); 6,6-dimethyl-2-methylenebicyclo[3.1.1]heptane (PINENE BETA); (4aS,8aR)-7-methyloctahydro-1,4-methanonaphthalen-6(2H)-one (PLICATONE); (1S,4R,6S)-4,7,7-trimethyl-4-(3-methylbut-2-en-1-yl)bicyclo[4.1.0]heptan-3-one (PRECARONE); 1-methyl-4-(4-methylpent-3-en-1-yl)cyclohex-3-enecarbaldehyde (PRECYCLEMONE B); benzyl propanoate (BENZYL PROPIONATE); ethyl propanoate (ETHYL PROPIONATE); (E)-3,7-dimethylocta-2,6-dien-1-yl propanoate (GERANYL PROPIONATE); (Z)-hex-3-en-1-yl propanoate (HEXENYL-3-CIS PROPIONATE); hexyl propanoate (HEXYL PROPIONATE); 3-methylbutyl propanoate (ISOAMYL PROPIONATE); (1 S)-1,7,7-trimethylbicyclo[2.2.1]heptan-2-yl propanoate (ISOBORNYL PROPIONATE); 3,7-dimethylocta-1,6-dien-3-yl propanoate (LINALYL PROPIONATE); 1-phenylethyl propanoate (STYRALLYL PROPIONATE); 2-(cyclohexylmethyl)-4,4,6-trimethyl-1,3-dioxane (RESEDAL); 2,4-dimethyl-4-phenyltetrahydrofuran (RHUBAFURAN); (2R,8aS)-3′,6-dimethyl-3,4,4a,5,8,8a-hexahydro-1H-spiro[1,4-methanonaphthalene-2,2′-oxirane](RHUBOFIX); (4aR,8aS,E)-6-ethylideneoctahydro-2H-5,8-methanochromene (RHUBOFLOR); 1,1-diethoxycyclohexane (RUM ACETAL); 4-methyl-2-(2-methylprop-1-en-1-yl)tetrahydro-2H-pyran (ROSE OXIDE); 2,2,2-trichloro-1-phenylethyl acetate (ROSACETOL); pentyl 2-hydroxybenzoate (AMYL SALICYLATE); cyclohexyl 2-hydroxybenzoate (CYCLOHEXYL SALICYLATE); ethyl 2-hydroxybenzoate (ETHYL SALICYLATE); 2-methylpropyl 2-hydroxybenzoate (ISOBUTYL SALICYLATE); methyl 2-hydroxybenzoate (METHYL SALICYLATE); 2-phenylethyl 2-hydroxybenzoate (PHENYL ETHYL SALICYLATE); 2′,2′,3,7,7-pentamethylspiro[bicyclo[4.1.0]heptane-2,5′-[1,3]dioxane](SPIRAMBRENE); (E)-6,10-dimethylundeca-5,9-dien-2-yl acetate (TANGERINOL); 1-methyl-4-propan-2-ylcyclohexa-1,3-diene (TERPINENE ALPHA); 1-methyl-4-propan-2-ylcyclohexa-1,4-diene (TERPINENE GAMMA); 3,7-dimethyloctanal (TETRAHYDRO CITRAL); 2,6-dimethyloctan-2-01 (TETRAHYDRO MYRCENOL); benzyl 2-methylbut-2-enoate (BENZYL TIGLATE); (E)-(E)-3,7-dimethylocta-2,6-dien-1-yl 2-methylbut-2-enoate (GERANYL TIGLATE); (Z)-hex-3-enyl](E)-2-methylbut-2-enoate (HEXENYL-3-CIS TIGLATE); (3E,5Z)-undeca-1,3,5-triene (UNDECATRIENE); (E)-4-methyldec-3-en-5-ol (UNDECAVERTOL); 5-methyloxolan-2-one (VALEROLACTONE GAMMA); (E)-2-ethoxy-5-(prop-1-en-1-yl)phenol (PROPENYL GUAETHOL, VANITROPE); 2,2,5-trimethyl-5-pentylcyclopentanone (VELOUTONE); (E)-methyl 2-((3-(4-(tert-butyl)phenyl)-2-methylprop-1-en-1-yl)amino)benzoate (VERDANTIOL); 1-methyl-4-(4-methylpentyl)cyclohex-3-enecarbaldehyde (VERNALDEHYDE); (2R,5R,8S)-4,4,8-trimethyltricyclo[6.3.1.02,5]dodecan-1-yl acetate (VETYNAL); [(3Z)-4,11,11-trimethyl-8-methylidene-5-bicyclo[7.2.0]undec-3-enyl]acetate (VETYVENAL); (2,2-dimethoxyethyl)benzene (VIRIDINE); (Z)-methyl 2-(3-oxo-2-(pent-2-en-1-yl)cyclopentyl)acetate (METHYL EPI JASMONATE); 2-methylpropyl (Z)-2-methylbut-2-enoate (ISOBUTYL ANGELATE); 4,4,8a-trimethyldecahydronaphthalen-4a-ol (METHYL GEOSMIN); ambreinol (CAS #73138-66-6); ambrette absolute; angelica seed oil; angelica root oil; anise oil; armoise oil; aspic oil; anise star oil; basil oil; copaiba balsam oil; peru balsam resinoid; tolu balsam resinoid; benzoin eco resinoid; bergamote oil; buchu leaf oil; camomille essence; cardamom seed oil; carrot seed oil; caraway oil; castoreum; cedar leaf oil; celery seed oil; beeswax absolute; cistus oil; citrodyle (CAS #68991-29-7); citron essence; citronella oil; citrus terpenes; civet artessence absolute; coriander oil; cornmint oil; costus; cumin oil; cypress oil; davana oil; elemi oil; olibanum oil; estragon oil; rum ether; Eucalyptus citriodora oil; eucalyptus oil; Eucalyptus globulus oil; fennel oil sweet; fir balsam oil; flouve odorante oil base; hay oil; galbanum oil; juniper oil; geranium oil; ginger oil; grapefruit oil; grapefruit terpenes; iracine 5/20; irival; jasmin absolute; jonquil hyacinth absolute; laurel noble oil; lavandin grosso oil; lime oil; magnolia flower oil; mandarin oil yellow; mandarin oil green; marjoram oil; mate absolute; melon honeydew; Mentha citrata; peppermint oil; peppermint terpenes; mimosa absolute; myrrh resinoid; myrtle oil; neroli oil; neroli phase essence; nutmeg oil; olibanol; orange aldehyde; orange oil bitter; orange oil; orange terpenes; peach white; parsley seed oil; petitgrain citronnier oil; fir oil; rosemary oil; rose absolute; rose de mai absolute; clary sage oil; spruce oil; tangerine oil; turpentine oil; thuja oil; vanilla absolute; vetyrisia; worm-wood oil; ylang ylang oil.

The present invention also provides a consumer product comprising the malodour counteracting fragrance composition of the present invention and a suitable carrier.

Consumer products include, but are not limited to, laundry products, such as fabric softeners, liquid detergents, or powder detergents; cosmetic products including personal care and hair care products, such as deodorants and antiperspirants, shampoo, conditioners, combing creams, leave on conditioners, styling cream, soaps, body creams and the like; and household cleaning products, such as all-purpose cleaners, for instance.

Depending on the intended application, the carrier may be a solvent, base or solid support, for instance.

The malodour counteracting fragrance composition of the present invention is particularly suitable for masking, reducing or preventing body malodour.

Therefore, in an embodiment, the consumer product of the present invention is a product for application to the human body, in particular a deodorant or antiperspirant product.

In this case, the carrier used in the consumer product should be suitable for application to the human body, i.e. cosmetically acceptable. Any carriers commonly used in the preparation of cosmetic preparations for use on the human skin may be employed in the present invention. Suitable carriers include, but are not limited to, liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like.

The consumer product may be produced in any solid, liquid, or semi-solid form useful for application to the skin. Thus, it may be a cream, an emulsion, a solution, an anhydrous composition, an aqueous dispersion, an oil, a milk, a balsam, a foam, an aqueous or oily lotion, an aqueous or oily gel, a slurry, an encapsulate, a hydro-alcoholic solution, a hydro-glycolic solution, a hydrogels, a liniment, a serum, a soap, an ointment, a pomade, a paste, a powder, a bar, a stick, flakes, beads, pellets, a spray or an aerosol (spray), including leave-on and rinse-off formulations.

Depending on the application, the consumer product of the present invention should contain more or less of the malodour counteracting fragrance composition of the present invention.

For instance, the concentration of the malodour counteracting fragrance composition in the consumer product may be from 0.1 wt % to 10 wt %, more preferably from 0.5 wt % to 5 wt %, and most preferably from 0.7 wt % to 3 wt %.

The consumer product of the present invention may further comprise one or more additives, such as other actives and/or adjuvants.

In an embodiment, the consumer product of the present invention further comprises skin care agents, soothing agents, deodorizing agent, probiotics, prebiotics, emollients, surfactants, thickening agents, sensates, polyols, humectants, polymers, silicones, enzymes, inorganic fillers, cationics, amphoterics, glycerides, esters, ethers, amino acids, polysaccharides, sugars, water or combinations thereof.

For instance, the consumer product of the present invention may comprise a skin care agent comprising a hydrophilic Phaeodactylum tricornutum extract. This cosmetic active, which has been described in WO 2020/178213 A1, has a soothing, comforting and inflammation-reducing effect on the skin and is therefore particularly advantageous for application to sensitive skin and sensitive skin areas. The disclosure of WO 2020/178213 A1 in this respect is herewith incorporated by reference.

In addition or alternatively, the consumer product of the present invention may also comprise a skin care agent comprising an Orobanche rapum extract. The Orobanche rapum extract comprises crenatoside and acteoside, which useful for balancing the skin microbiota, as described in WO 2018/185060 A1. The disclosure of WO 2018/185060 A1 in this respect is herewith incorporated by reference. Combining the malodour counteracting fragrance composition of the present invention with an Orobanche rapum extract provides a particularly microbiome-friendly consumer product.

The consumer product of the present invention may also advantageously comprise one or more deodorant or antiperspirant actives. This will allow to further increase the malodour counteractancy of the fragrance composition of the present invention.

For instance, the consumer product of the present invention may comprise a deodorizing agent comprising a zinc carboxylate salt. Zinc carboxylate salts, and in particular zinc neodecanoate, are able to deliver a deodorancy benefit and a sustained, undistorted and pleasant scent impression, as described in WO 2018/087147 A1. It has also been found to improve the efficacy and long-lastingness of a deodorant composition. The disclosure of WO 2018/087147 A1 in this respect is herewith incorporated by reference.

In addition or alternatively, the consumer product of the present invention may also comprise a deodorizing agent comprising a compound of formula (I)

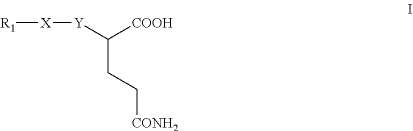

wherein Y represents a direct bond to X, or a divalent chain that may contain carbon, oxygen or nitrogen atoms, X is a zinc-chelating group, and R₁ is a linear, branched or cyclic carbon chain having about 1 to 14 carbon atoms, optionally containing one or more heteroatoms such as O, N or S, or unsaturation, the chain optionally supporting one or more substituents selected from amide, ester, keto, ether, amine, halogen or hydroxyl, or aryl or heteroaryl groups, which aryl or heteroaryl groups optionally support one or more substituents selected from amide, ester, keto, ether, amine, halogen, alkyl and/or hydroxyl. This kind of deodorizing agents has been described in EP 1 258 531, which is herewith incorporated by reference.

The consumer product of the present invention may also comprise one or more prebiotics.

For instance, the consumer product of the present invention may comprise a prebiotic selected from the group consisting of fructans, galactans, pectin, beta-glucans, and combinations thereof. A number of prebiotics, which are able to balance the skin microbiome and are therefore advantageously included in the consumer product of the present invention, are described in WO 2020/052916. The disclosure of WO 2020/052916 and of the documents cited therein related to such prebiotics is herewith incorporated by reference.

The present invention also relates to a method of masking, reducing and/or preventing body malodour, said method comprising the step of topically applying the malodour counteracting fragrance composition or the consumer product of the present invention to the human body.

In particular, the fragrance composition or consumer product may be applied to a location where body malodour is generally formed, for instance the underarm area, feet, scalp, crotch or groin.

The present invention is further illustrated by means of the following non-limiting examples:

### EXAMPLE 1: BACTERIOSTATIC ACTIVITY OF FRAGRANCE INGREDIENTS

The axilla test bacteria Corynebacterium Ax15 was isolated from the human axilla according to techniques known in the art and according to standard microbiological practice, and as it has been described in the literature (Natsch, A. et al., J. Biol. Chem., 2003, 278 (8): p. 5718-27). The strain was maintained on Tryptic soy broth plates, this standard medium being supplemented with 5 g per litre of Tween 80. Plates were incubated at 36° C. for a period 48 hours. The bacteria were then swabbed from the plates and suspended in 4 ml of Müller-Hinton broth supplemented with 100 mg of Tween 80 per litre (MH-Tween) and incubated again at 36° C. for another 16 hours. Following incubation, the bacterial suspensions were diluted in MH-Tween to obtain a final cell density of 107 colony forming units per ml. These diluted suspensions of the test organism were distributed to different wells of a microtiter plate at 100 μl per well.

The fragrance ingredients to be tested were dissolved in dimethylsulfoxide (DMSO) at various test concentrations, and 2.5 μl each of these DMSO solutions were added to the wells of the test plates to reach final concentrations of 0.05-0.2%, depending on dilution. The plates were covered with plastic film and incubated for 24 h at 36° C. with shaking at 250 rpm.

The turbidity developing in the microtiter plates was then examined after 24 h to determine microbial growth.

If the turbidity of the test organism is inhibited by at least 80%, the compound is rated as antibacterial at that concentration.

### EXAMPLE 2: SENSORY TESTING

The following fragrance compositions were included at a concentration of 1.0% w/w in the aerosol deodorants:

| | Fragrance | Fragrance | Fragrance | Fragrance | Fragrance | Fragrance | |
|---|---|---|---|---|---|---|---|
| Ingredient | 1 [wt %] | 2 [wt %] | 3 [wt %] | 4 [wt %] | 5 [wt %] | 6 [wt %] | Group |
| BENZYL ACETATE | 1.5 | 1 | | | 0.33 | 0.3 | |
| CEDRYL ACETATE | | | | | 1.11 | | |
| CITRONELLYL ACETATE | 6 | 1 | | | | | |
| DIMETHYL BENZYL | 9 | | | | | | |
| CARBINYL ACETATE | | | | | | | |
| GERANYL ACETATE | 13 | | | | | | |
| HEXENYL-3-CIS | 0.15 | | 0.29 | 0.27 | | | |
| ACETATE | | | | | | | |
| HEXYL ACETATE | 0.6 | 0.8 | | | | | |
| LINALYL ACETATE | 9 | 2 | 1.43 | | 5.56 | 5 | |
| PHENYL ETHYL | 1.5 | | | | | | |
| ACETATE | | | | | | | |
| BUTYL CYCLOHEXYL | | | 1.43 | 2.67 | | | |
| ACETATE PARA | | | | | | | |
| AGRUMEX | | | | 0.67 | | | C |
| PHENYL ETHYL | 15 | 4 | | | | | |
| ALCOHOL | | | | | | | |
| AMYL CINNAMIC | | 10 | | | 8.44 | | C |
| ALDEHYDE | | | | | | | |
| HEXYL CINNAMIC | | 3 | | | | 5.9 | C |
| ALDEHYDE | | | | | | | |
| ALDEHYDE C 110 | | | 0.1 | 0.13 | | | D |
| UNDECYLIC | | | | | | | |
| ALDEHYDE C 12 LAURIC | | | | 0.03 | | | D |
| ALDEHYDE C 12 MNA | | | | 0.01 | | | D |
| ALLYL AMYL | | 0.5 | 0.29 | 0.27 | | | |
| GLYCOLATE | | | | | | | |
| AMBER CORE | | | | 1.33 | | | D |
| AMBROFIX FLAKES | | 0.5 | | 0.67 | | 2 | |
| AUBEPINE PARA | | 1 | | | | | |
| CRESOL | | | | | | | |
| BERGAMOT OIL | | 4 | | | | | |
| BERGAMOT BASE | | | | | 11.11 | 20.6 | |
| BERRYFLOR | 0.3 | | | | | | |
| ETHYLENE | | | | 2 | | 2.8 | |
| BRASSYLATE | | | | | | | |
| CALONE | | 0.3 | 0.01 | 0.04 | | | |
| CAMPHOR | | | 0.21 | 0.27 | | | |
| ALLYL CAPRONATE | | 0.2 | | | | | |
| CARVONE LAEVO | | 0.1 | 0.29 | 0.33 | | | |
| CASHMERAN | | 1 | 0.57 | 0.53 | | | |
| CEDRYL METHYL | | | 1.43 | 1.33 | | | |
| ETHER | | | | | | | |
| LEMON OIL ITALIAN | | | | | 1.11 | 7 | C |
| CITRONELLOL EXTRA | | | | | 5.56 | | D |
| COUMARINE PURE | | | 0.14 | 0.13 | | | D |
| CRYSTALS | | | | | | | |
| CYCLOGALBANATE | | 1 | 0.14 | 0.16 | | | |
| DAMASCENONE | | | 0.01 | | | | D |
| DAMASCONE ALPHA | | | | 0.13 | | | B |
| DAMASCONE DELTA | | 0.05 | | | | | A |
| DECALACTONE DELTA | 1.5 | 0.06 | | | | | |
| METHYL DIHYDRO | | | 14.29 | | 5.56 | | |
| ISOJASMONATE | | | | | | | |
| DIHYDRO MYRCENOL | | 1 | | | 7.78 | 2 | |
| DIMETHYL PHENYL | 9 | | | | | | |
| ETHYL CARBINOL | | | | | | | |
| ETHYL LINALOOL | 9 | 3.5 | | | | | |
| ETHYL MALTOL 1%/AB | | | | | 0.11 | | |
| EUCALYPTUS | | | | | 0.22 | 0.2 | |
| GLOBULUS OIL | | | | | | | |
| FLORALOZONE | | | 0.43 | 0.27 | | | D |
| FLOROCYCLENE | | | | 1.33 | | | B |
| FLOROSA | | | | | 6.67 | | C |
| OXYOCTALINE | | | | 2.67 | | | D |
| FORMATE | | | | | | | |
| STRAWBERRY PURE | 0.3 | | | | | | |
| FRESKOMENTHE | | | 0.43 | 0.4 | | | |
| GALBANONE (10% in | | | 0.29 | 1.07 | | | C |
| dipropylene glycol) | | | | | | | |
| GARDENOL | | 2.5 | | | | | |
| GEORGYWOOD | | 0.1 | | | | | A |
| GERANIOL | | | | | 2.22 | | |
| GERANIUM BASE | 1.2 | | | | | | |
| GIVESCONE | | | 0.07 | 0.07 | | | |
| HABANOLIDE | | | | 1.33 | | | |
| HEDIONE | | | | 40 | | 20 | A |
| HELIOTROPINE | | 2 | | | | | C |
| CRYSTALS | | | | | | | |
| HEXENOL-3-CIS | 0.15 | 0.3 | | | | | |
| HYDROXYCITRONELLAL | | | 1.43 | | | | D |
| IRALIA | | 1 | | | | | |
| IRISANTHEME | | 1.5 | | | | | |
| IRISONE ALPHA | 1.5 | | | | | | C |
| ISO E SUPER | | 1 | 42.86 | 16.17 | | | |
| ISOJASMONE B 11 | 0.15 | | | | | | |
| ISORALDEINE 70 | | 1 | 1.43 | 1.33 | | | B |
| JASMONYL | | 0.2 | | | | | |
| LAVANDIN GROSSO OIL | | | 1.43 | 0.67 | 0.33 | | |
| LEMONGRASS | | | | | 0.56 | | |
| ESSENCE | | | | | | | |
| LIME OIL | | | | | 4.44 | 4 | |
| LINALOOL | | 10 | 7.14 | | 13.33 | | |
| LITSEA CUBEBA OIL | | | | | 0.56 | | D |
| MAHONIAL | | | | | 0.56 | 0.5 | |
| MANZANATE | | 0.02 | 0.03 | 0.04 | | | |
| MELONAL | | 0.2 | | | | | |
| SPEARMINT OIL | | | | | 0.44 | 0.4 | D |
| METHOXY PHENYL | 0.3 | | | | | | |
| BUTANONE | | | | | | | |
| METHYL | 3 | | | | | | |
| PAMPLEMOUSSE | | | | | | | |
| MOXALONE 50%/DPG | 1.5 | | | | | | |
| MUSK C14 | | | 2.86 | | | | |
| NEROLEX | | | | | 1.67 | 1.5 | |
| ORANGE OIL | | | | | 12.22 | 2 | |
| ORANGE TERPENES | | 3 | | | | | |
| PATCHOULI OIL | | | 0.14 | 0.67 | | | D |
| PEACH PURE | | | | | 0.22 | 0.3 | D |
| PELARGOL | | 8 | | | | 10 | C |
| PEONILE | | 1 | | 0.27 | | | B |
| PHARAONE 10%/DPG | | | 0.57 | 0.67 | | | |
| PINENE BETA | | | | | 1.11 | | |
| RHUBAFURAN | 0.03 | | | | | | |
| ROSE OXIDE | 0.15 | | 0.07 | 0.08 | | | |
| ROSYFOLIA | | | | | | 0.5 | |
| AMYL SALICYLATE | 16.17 | 2 | | | | | |
| BENZYL SALICYLATE | | 25.46 | | 13.33 | | 7.5 | A |
| CYCLOHEXYL | | 1 | | | | | |
| SALICYLATE | | | | | | | |
| HEXENYLE-3-CIS | | 0.6 | | 2.67 | | 3 | |
| SALICYLATE | | | | | | | |
| HEXYL SALICYLATE | | | 20.16 | | 5.56 | | C |
| SYLKOLIDE | | | | | 2.22 | 4 | |
| THIBETOLIDE | | 3 | | | | | C |
| TRICYCLAL | | | | | 0.56 | 0.5 | D |
| UNDECAVERTOL | | 0.1 | | | | | |
| BERGAMOT OIL GREEN | | | | | 0.22 | | |
| MANDARINE OIL GREEN | | | | | 0.22 | | |
| VERTOFIX COEUR | | 1 | | 6 | | | C |
| YARA YARA | | 0.01 | | | | | C |
| Score (5 × Group | 4.5 | 216 | 67.1 | 302 | 65 | 208 | |
| A + 4 × Group B + | | | | | | | |
| 3 × Group C) | | | | | | | |

Six deodorant sprays each containing a different fragrance composition (Fragrances 1 through 6) were self-assessed by naïve volunteers in three separate tests. In each of the three tests, two fragrance compositions having a similar odour type were applied in the morning and then compared in the evening with regard to their sensory performance.

Fragrances 2, 4, and 6 are fragrance compositions according to the present invention, whereas Fragrances 1, 3, and 5 are comparative examples.

Deodorant Products

The fragrance compositions were incorporated in a standard deodorant base with the following concentrations:

| | | | Concentration |
|---|---|---|---|
| Constituent | Supplier | INCI Name | [w/w] |
| Eutanol G | Cognis | Octyl dodecanol | 2.5% |
| Ethyl alcohol 96% | | Ethyl alcohol | 90% |
| Propylene glycol | Prod'Hyg | Propylene glycol | 5% |
| Fragrance composition | Givaudan | | 2.5% |

All constituents were mixed and then filled into a deodorant spray can. Filling ratio: 40% of the above deodorant composition and 60% of gas (propane/butane blend).

Test 1:

Deodorant sprays 1 and 2 were coded and provided to five (5) naïve volunteers. The volunteers were asked to wash as normal and then, on the morning of the test, to apply one deodorant to one underarm and the other deodorant to the other underarm. The products were randomised (left/right underarm) across the volunteers.

The volunteers then went about their day as normal. At the end of the day (˜10 hours after application), the volunteers were asked, “Which arm has less malodour?”

Result: Four out of the five volunteers selected the deodorant containing fragrance 2.

Test 2:

Deodorant sprays 3 and 4 were coded and provided to approximately sixty (60) naïve volunteers. The volunteers were asked to wash as normal and then, on the morning of the test, to apply one deodorant to one underarm and the other deodorant to the other underarm. The products were randomised (left/right underarm) across the volunteers.

The volunteers then went about their day and normal. At the end of the day (˜10 hours after application), the volunteers were asked, “Which underarm feels the freshest after wearing the product all day?”

Result: The underarms treated with deodorant spray 4, containing the fragrance composition of the present invention, were perceived as significantly (90% confidence level) fresher than those treated with deodorant spray 3, which contained the comparative fragrance.

Test 3:

Deodorant sprays 5 and 6 were coded and provided to approximately sixty (60) naïve volunteers. The volunteers were asked to wash as normal and then, on the morning of the test, to apply one deodorant to one underarm and the other deodorant to the other underarm. The products were randomised (left/right underarm) across the volunteers.

The volunteers then went about their day and normal. At the end of the day (˜10 hours after application), the volunteers were asked to score each underarm for perceived malodour intensity on a scale of 1 to 10 (1 being low/no malodour and 10 being high malodour).

Result: The underarms treated with deodorant spray 6, containing the fragrance composition of the present invention, were perceived as having significantly (95% confidence level) lower intensity of malodour than those treated with deodorant spray 5, which contained the comparative fragrance.

1. A malodour counteracting fragrance composition comprising one or more fragrance ingredients drawn from Group A and/or one or more fragrance ingredients drawn from Group B and/or one or more fragrance ingredients drawn from Group C, wherein the concentrations of said fragrance ingredients are selected such that
5×[total concentration Group A]+4×[total concentration Group B]+3×[total concentration Group C]≥100;
wherein [total concentration Group A] is the sum of the individual concentrations of all fragrance ingredients drawn from Group A, and [total concentration Group B] is the sum of the individual concentrations of all fragrance ingredients drawn from Group B, and [total concentration Group C] is the sum of the individual concentrations of all fragrance ingredients drawn from Group C, all concentrations being indicated in wt % based on the total weight of the malodour counteracting fragrance composition;
wherein the Group A fragrance ingredients are selected from the group consisting of 3,8,8,11a-tetramethyldodecahydro-1H-3,5a-epoxynaphtho[2,1-c]oxepine (AMBERKETAL); (3aR,5aS,9aS,9bR)-3a,6,6,9a-tetramethyl-2,4,5,5a,7,8,9,9b-octahydro-1H-benzo[e][1]benzofuran (AMBROFIX, AMBROXAN); 3a,6,6,9a-tetramethyl-2,4,5,5a,7,8,9,9b-octahydro-1H-benzo[e][1]benzofuran (CETALOX); (E)-1-(2,6,6-trimethyl-1-cyclohexenyl)but-2-en-1-one (DAMASCONE BETA); 1-(2,6,6-trimethyl-1-cyclohex-3-enyl)but-2-en-1-one (DAMASCONE DELTA); 3-methyl-2-pentylcyclopent-2-enone (DIHYDRO JASMONE); 1-(1,2,8,8-tetramethyl-1,2,3,4,5,6,7,8-octahydronaphthalen-2-yl)ethanone (GEORGYWOOD); methyl 3-oxo-2-pentylcyclopentaneacetate (HEDIONE, CEPIONATE); benzyl 2-hydroxybenzoate (BENZYL SALICYLATE); (Z)-hex-3-en-1-yl 2-hydroxybenzoate (HEXENYL-3-CIS SALICYLATE); (E)-tridec-2-enenitrile (TRIDECENE-2-NITRILE); and mixtures thereof;
wherein the Group B fragrance ingredients are selected from the group consisting of (E)-3-methyl-4-(2,6,6-trimethylcyclohex-2-en-1-yl)but-3-en-2-one (ISORALDEINE CETONE ALPHA, ALPHA ISOMETHYL IONONE); benzyl 3-phenylprop-2-enoate (BENZYL CINNAMATE); methyl 3-phenylprop-2-enoate (METHYL CINNAMATE); (E)-1-(2,6,6-trimethylcyclohex-2-en-1-yl)but-2-en-1-one (DAMASCONE ALPHA); 3-ethoxy-4-hydroxybenzaldehyde (ETHYL VANILLIN); 2-(phenoxy)ethyl 2-methylpropanoate (PHENOXY ETHYL ISOBUTYRATE); mixtures of isomers (E)- and (Z)-3-methyl-4-(2,6,6-trimethyl-2-cyclohexen-1-yl)-3-buten-2-one, and (E)-1-(2,6,6-trimethylcyclohex-2-en-1-yl)pent-1-en-3-one (ISORALDEINE 70, ISORALDEINE 95); 4-(4-hydroxyphenyl)butan-2-one (RASPBERRY KETONE); 2-(2-(4-methylcyclohex-3-en-1-yl)propyl)cyclopentan-1-one (NECTARYL); 2-cyclohexylidene-2-phenylacetonitrile (PEONILE); (3aR,6S,7aS)-3a,4,5,6,7,7a-hexahydro-1H-4,7-methanoinden-6-yl propanoate (FLOROCYCLENE); ethyl 2,6,6-trimethylcyclohexa-1,3-diene-1-carboxylate (ETHYL SAFRANATE); 1-((2E,5Z,9Z)-2,7,8-trimethylcyclododeca-2,5,9-trien-1-yl)ethanone (TRIMOFIX O); and mixtures thereof;
wherein the Group C fragrance ingredients are selected from the group consisting of 2-(4-methyl-1-cyclohex-3-enyl)propan-2-yl acetate (TERPINYL ACETATE); (3aR,6S,7aS)-3a,4,5,6,7,7a-hexahydro-1H-4,7-methanoinden-6-yl acetate (JASMACYCLENE); 2-(tert-butyl)cyclohexyl acetate (AGRUMEX); (E)-3-phenylprop-2-en-1-ol (CINNAMIC ALCOHOL); (Z)-2-benzylideneheptanal (AMYL CINNAMIC ALDEHYDE); (E)-2-benzylideneoctanal (HEXYL CINNAMIC ALDEHYDE); (Z)-2-methyl-3-phenylacrylaldehyde (METHYL CINNAMIC ALDEHYDE); methyl 2-aminobenzoate (METHYL ANTHRANILATE); 4-phenylbutan-2-one (BENZYL ACETONE); (1S,2R,5R)-2-ethoxy-2,6,6-trimethyl-9-methylenebicyclo[3.3.1]nonane (BOISIRIS); 3-methyl-5-propylcyclohex-2-enone (CELERY KETONE); 2-pentylcyclopentanone (QUINTONE); oxydibenzene (DIPHENYL OXIDE); tetrahydro-4-methyl-2-(2-methylpropyl)-2H-pyran-4-ol (FLOROSA); 1-(5,5-dimethylcyclohex-1-en-1-yl)pent-4-en-1-one (GALBANONE); 2-(2-hydroxypropan-2-yl)-5-methylcyclohexanol (GERANODYLE); 2-(8-isopropyl-6-methylbicyclo[2.2.2]oct-5-en-2-yl)-1,3-dioxolane (GLYCOLIERRAL); 3a-ethyl-6,6,9a-trimethyldodecahydronaphtho[1,2-c]furan (GRISALVA); benzo[d][1,3]dioxole-5-carbaldehyde (HELIOTROPINE CRYSTALS); (E)-4-(2,6,6-trimethylcyclohex-2-en-1-yl)but-3-en-2-one (IRISONE ALPHA); (2E,6Z)-3,7-dimethylnona-2,6-dienenitrile (LEMONILE); 4-methyl-1-propan-2-ylbicyclo[2.2.2]oct-2-ene-8-carboxylate (MAHAGONATE); (2-butan-2-yl-1-methylcyclohexyl) acetate (METAMBRATE); methyl 2-(methylamino)benzoate (DIMETHYL ANTHRANILATE); 1-((1S,8aS)-1,4,4,6-tetramethyl-2,3,3a,4,5,8-hexahydro-1H-5,8a-methanoazulen-7-yl)ethanone (METHYL CEDRYL KETONE); (Z)-3-methylcyclopentadec-5-enone (MUSCENONE); 2-ethoxynaphthalene (NEROLINE CRYSTALS); 2,4-dimethyl-2-(5,5,8,8-tetramethyl-5,6,7,8-tetrahydronaphthalen-2-yl)-1,3-dioxolane (OKOUMAL); 3,7-dimethyloctan-1-ol (PELARGOL); (E)-3,7-dimethylocta-2,6-dien-1-yl 2-phenylacetate (GERANYL PHENYL ACETATE); 2-phenylethyl 2-phenylacetate (PHENYL ETHYL PHENYL ACETATE); 5-pentyldihydrofuran-2(3H)-one (PRUNOLIDE); hexyl 2-hydroxybenzoate (HEXYL SALICYLATE); 1-(spiro[4.5]dec-7-en-7-yl)pent-4-en-1-one (SPIROGALBANONE); 3,7-dimethyloctan-3-ol (TETRAHYDRO LINALOOL); oxacyclohexadecan-2-one (THIBETOLIDE); 4-hydroxy-3-methoxybenzaldehyde (VANILLIN); 1-((1S,8aS)-1,4,4,6-tetramethyl-2,3,3a,4,5,8-hexahydro-1H-5,8a-methanoazulen-7-yl)ethanone (VERTOFIX COEUR); 2-methoxynaphtalene (YARA YARA); lemon oil, petitgrain oil, sage officinalis oil, and mixtures thereof; and
wherein the malodour counteracting fragrance composition may optionally comprise additional fragrance ingredients, provided that the total concentration of fragrance ingredients selected from Group D does not exceed 10 wt % based on the total weight of the malodour counteracting fragrance composition,
wherein the Group D fragrance ingredients are selected from the group consisting of ixtures of 3,7-dimethyloct-6-en-1-yl acetate and 3,7-dimethyloct-7-en-1-yl acetate (RHODINYL ACETATE); 4,8-dimethyl-2-propan-2-ylidene-3,3a,4,5,6,8a-hexahydro-1H-azulen-6-yl acetate (VETIVERYL ACETATE); (E)-2-methoxy-4-(prop-1-en-1-yl)phenyl acetate (ACETYL ISOEUGENOL); 2-phenylacetic acid (PHENYL ACETIC ACID); 2,6,10-trimethylundec-9-enal (ADOXAL); decan-1-ol (ALCOHOL C 10 DECYLIC); 10-UNDECEN-1-OL (ALCOHOL C 11 UNDECYLENIC); dodecan-1-ol (ALCOHOL C 12 LAURIC); tridecan-1-ol (ALCOHOL C 13 OXO); octan-1-ol (ALCOHOL C 8 OCTYLIC); nonan-1-ol (ALCOHOL C 9 NONYLIC); (4-isopropylphenyl)methanol (CUMINYL ALCOHOL); (4-prop-1-en-2-yl-1-cyclohexenyl)methanol (PERILLA ALCOHOL); decanal (ALDEHYDE C 10 DECYLIC); undecanal (ALDEHYDE C 110 UNDECYLIC); hexanal (ALDEHYDE C 6 HEXYLIC); heptanal (ALDEHYDE C 7 HEPTYLIC); nonanal (ALDEHYDE C 9 NONYLIC); (2E)-3-phenylprop-2-enal (CINNAMIC ALDEHYDE); 2-phenylpropanal (HYDRATROPIC ALDEHYDE); 3-phenylpropanal (PHENYL PROPIONIC ALDEHYDE); (2R,8aS)-1-[(E)-6-[(1S)-2,2-dimethyl-6-methylidenecyclohexyl]-4-methylhex-3-enyl]-2,5,5,8a-tetramethyl-3,4,4a,6,7,8-hexahydro-1H-naphthalen-2-ol (AMBREINE); methyl 2-(benzylideneamino)benzoate (AMANDOLENE); (E)-methyl 2-((7-hydroxy-3,7-dimethyloctylidene)amino)benzoate (AURANTIOL); 6-isopropylquinoline (ISOPROPYL QUINOLINE); 2-(2-methylpropyl)quinoline (BUTYL QUINOLINE SECONDARY); (E)-3,7-dimethylocta-2,6-dien-1-yl butanoate (GERANYL BUTYRATE); (Z)-hex-3-en-1-yl butanoate (HEXENYL-3-CIS BUTYRATE); (4Z,8Z)-1,5,9-trimethyl-13-oxabicyclo[10.1.0]trideca-4,8-diene (CEDROXYDE); (E)-1-(2,6,6-trimethylcyclohex-2-en-1-yl)hepta-1,6-dien-3-one (CETONE V); (Z)-1,1-diethoxy-3,7-dimethylocta-2,6-diene (CITRATHAL R); 3,7-dimethyloct-6-en-1-ol (CITRONELLOL EXTRA); 3,7-dimethyloct-6-enenitrile (CITRONELLYL NITRILE); 2-((3,7-dimethyloct-6-en-1-yl)oxy)acetaldehyde (CITRONELLYL OXYACETALDEHYDE); 2-methoxy-4-methylphenol (CREOSOL); 2,4-dimethylcyclohex-3-ene-1-carbaldehyde (CYCLAL C); 3-(4-methylcyclohex-3-en-1-yl)butan-1-ol (CYCLOMETHYLENE CITRONELLOL); 8,8-dimethyl-1,2,3,4,5,6,7,8-octahydronaphthalene-2-carbaldehyde (CYCLOMYRAL); (E)-1-(2,6,6-trimethylcyclohexa-1,3-dien-1-yl)but-2-en-1-one (DAMASCENONE); 5-hexyloxolan-2-one (DECALACTONE GAMMA); 2-methoxy-4-propylphenol (DIHYDRO EUGENOL); 2-(4-methylcyclohexyl)propan-2-ol (DIHYDRO TERPINEOL); 2-(2-(3,3,5-trimethylcyclohexyl)acetyl)cyclopentanone (DIONE); 6-heptyltetrahydro-2H-pyran-2-one (DODECALACTONE DELTA); 5-octyloxolan-2-one (DODECALACTONE GAMMA); 4-allyl-2-methoxyphenol (EUGENOL); (2E,6Z)-3,7,11-trimethyldodeca-2,6,10-trien-1-ol (FARNESOL); 1-(3,5,5,6,8,8-hexamethyl-5,6,7,8-tetrahydronaphthalen-2-yl)ethanone (FIXOLIDE); 3-(4-ethylphenyl)-2,2-dimethylpropanal (FLORALOZONE); 4,4,8,8-tetramethyloctahydro-4a,7-methanonaphtho[1,8a-b]oxirene (FOLENOX); 4-isopropylcyclohexanol (FOLROSIA); 4,6,6,7,8,8-hexamethyl-1,3,4,6,7,8-hexahydrocyclopenta[g]isochromene (GALAXOLIDE); (E)-3,7-dimethylocta-2,6-dien-1-ol (GERANIOL); mixtures of thioterpenes (GRAPELIONE); methyl 7-isopropyl-1,4a-dimethyl-1,2,3,4,4a,4b,5,6,7,8,10,10a-dodecahydrophenanthrene-1-carboxylate (HERCOLYN); (Z)-hex-3-en-1-yl (Z)-hex-3-enoate (HEXENYL-3-CIS HEXENOATE); 7-hydroxy-3,7-dimethyloctanal (HYDROXYCITRONELLAL); 8,8-di(1H-indol-3-yl)-2,6-dimethyloctan-2-ol (INDOLENE); 6-butan-2-yl-quinoline (ISOBUTYL QUINOLINE-2); 3,7-dimethylocta-1,6-dien-3-yl 2-methylpropanoate (LINALYL ISOBUTYRATE); 2,4,6-trimethylcyclohex-3-enecarbaldehyde (ISOCYCLOCITRAL); (Z)-3-methyl-2-(pent-2-en-1-yl)cyclopent-2-enone (JASMONE CIS); (Z)-5-(hex-3-en-1-yl)-5-methyloxolan-2-one (JASMIN LACTONE GAMMA); 2,8,8-trimethyloctahydro-1H-4a,2-(epoxymethano)naphthalen-10-one (LACTOSCATONE); 8-isopropyl-1-oxaspiro[4.5]decan-2-one (LAITONE); (E)-3,7-dimethylocta-2,6-dienal (CITRAL); 3-hydroxy-2-methyl-4H-pyran-4-one (MALTOL); 4-[(3E)-4,8-dimethylnona-3,7-dienyl]pyridine (MARITIMA); 3-methyl-5-phenylpentanal (MEFRANAL); 5-hexyl-5-methyloxolan-2-one (METHYL DECALACTONE GAMMA); (E)-1,2-dimethoxy-4-(prop-1-en-1-yl)benzene (METHYL ISOEUGENOL); 2-methyldecanal (ALDEHYDE C 11 MOA); 4-(4-methylpent-3-en-1-yl)cyclohex-3-enecarbaldehyde (MYRALDENE); 3,7-dimethyl-2,6-octadien-1-ol (NEROL C); (Z)-3,7,11-trimethyldodeca-1,6,10-trien-3-ol (NEROLIDOL); 6,8-dimethylnonan-2-ol (NONADYL); 4,4a-dimethyl-6-(prop-1-en-2-yl)-4,4a,5,6,7,8-hexahydronaphthalen-2(3H)-one (NOOTKATONE CRYSTALS); (E)-3,7-dimethylocta-1,3,6-triene (OCIMENE); 3-methoxy-5-methylphenol (ORCINYL 3); p-cresol (CRESOL PARA); 6-methylquinoline (METHYL QUINOLINE PARA); 2-(6,6-dimethyl-5-bicyclo[2.2.1]heptanylidene)ethanol (PATCHOMINT); 2-phenoxyacetaldehyde (PHENOXY ACETALDEHYDE); 3-(6,6-dimethylbicyclo[3.1.1]hept-2-en-2-yl)propanal (PINOACETALDEHYDE); 3,7-dimethyloct-6-en-1-yl propanoate (CITRONELLYL PROPIONATE); 2-ethoxy-4-(isopropoxymethyl)phenol (PROPYL DIANTILIS); 6-(sec-butyl)quinoline (PYRALONE); mixtures of 3,7-dimethyloct-6-en-1-ol and 3,7-dimethyloct-7-en-1-ol (RHODINOL); 2-methyl-5-phenylpentan-1-ol (ROSAPHEN); 3-methyl-5-(2,2,3-trimethylcyclopent-3-en-1-yl)pentan-2-ol (SANDALORE); (3aR,4R,6S,7R,7aR)-6-methoxyoctahydro-1H-4,7-methanoindene-1-carbaldehyde (SCENTENAL); (E)-5-methylheptan-3-one oxime (STEMONE); (E)-6-ethyl-3-methyloct-6-en-1-ol (SUPER MUGUET); 1-methyl-4-(propan-2-ylidene)cyclohex-1-ene (TERPINOLENE); 3-(2,6,6-trimethyl-1-cyclohexen-1-yl)-prop-2-enenitrile (TERRANIL); 2,2,6-trimethylcyclohexyl-3-hexanol (TIMBEROL); 3-(benzo[d][1,3]dioxol-5-yl)-2-methylpropanal (TROPIONAL); 4-methyl-5-pentyldihydrofuran-2(3H)-one (METHYL TUBERATE); 6-hexyltetrahydro-2H-pyran-2-one (UNDECALACTONE DELTA); (Z)-cyclohexadec-5-enone (VELVIONE); 2-(tert-butyl)cyclohexanol (VERDOL); 2-ethyl-4-hydroxy-5-methylfuran-3(2H)-one (HOMOFURONOL); (Z)-non-6-en-1-ol (NONENOL-6-CIS); (2Z,6E)-2,6-nonadien-1-yl acetate (NONADIENYL ACETATE); (4-methylphenyl) acetate (PARA CRESYL ACETATE); (4S)-4,7,7-trimethyl-6-thiabicyclo[3.2.1]octane (CORPS PAMPLEMOUSSE); 2-(3-phenylpropyl)pyridine (CORPS RACINE VS); (2E,4E)-deca-2,4-dienal (DECADIENAL); (E)-dec-2-enal (DECENAL-2-TRANS); (E)-dodec-2-enal (DODECENAL); 8-methyl-1-oxaspiro[4.5]decan-2-one (METHYL LAITONE); (2E,6Z)-nona-2,6-dienal (LIME DIENES, NONADIENAL); (2Z,6E)-2,6-nonadien-1-ol (NONADIENOL-2,6); (Z)-non-6-enal (NONENAL-6-CIS); 4-ethyl-5-methylthiazol-2-ol (HYDROXY ETHYL METHYL THIAZOL); 2,6,6-trimethylcyclohexa-1,3-dienecarbaldehyde (SAFRANAL); 1-bicyclo [2.2.1]hept-5-en-2-yl-ethanone oxime (TERRAVERT); (E)-dodec-2-enal (ALDEHYDE MANDARINE); undec-10-enal (ALDEHYDE C 11 UNDECYLENIC); dodecanal (ALDEHYDE C 12 LAURIC); 2-methylundecanal (ALDEHYDE C 12 MNA); 3-(4-isopropylphenyl)-2-methylpropanal (CYCLAMEN ALDEHYDE); (E)-undec-9-enal (ALDEHYDE ISO C 11); 1-((2-(tert-butyl)cyclohexyl)oxy)butan-2-ol (AMBER CORE); 3-(4-(tert-butyl)phenyl)propanal (BOURGEONAL); 5-isopropyl-2-methylphenol (CARVACROL); 2H-chromen-2-one (COUMARIN CRYSTALS); 4-(4-hydroxy-4-methylpentyl)cyclohex-3-enecarbaldehyde (CYCLOHEXAL); (E)-dec-4-enal (DECENAL-4-TRANS); (E)-4-((3aS,7aS)-hexahydro-1H-4,7-methanoinden-5(6H)-ylidene)butanal (DUPICAL); (E)-3-methyl-5-(2,2,3-trimethylcyclopent-3-en-1-yl)pent-4-en-2-ol (EBANOL); 2-ethyl-3-hydroxy-4H-pyran-4-one (ETHYL MALTOL); methyl 2,4-dihydroxy-3,6-dimethylbenzoate (EVERNYL); 3-(4-methoxyphenyl)-2-methylpropanal (FENNALDEHYDE); 3a,6,6,9a-tetramethyldodecahydronaphtho[2,1-b]furan (FIXAMBRENE); 3-(3-isopropylphenyl)butanal (FLORHYDRAL); 2,4a,5,8a-tetramethyl-1,2,3,4,4a,7,8,8a-octahydronaphthalen-1-yl formate (OXYOCTALINE FORMATE); (E)-4-(2,6,6-trimethylcyclohex-1-en-1-yl)but-3-en-2-one (IONONE BETA); 4-formyl-2-methoxyphenyl 2-methylpropanoate (ISOBUTAVAN); (E)-2-methoxy-4-(prop-1-en-1-yl)phenol (ISOEUGENOL); (1-methyl-2-((1,2,2-trimethylbicyclo[3.1.0]hexan-3-yl)methyl)cyclopropyl)methanol (JAVANOL); 3-(4-(tert-butyl)phenyl)-2-methylpropanal (LILIAL); 2,2-dimethyl-3-(m-tolyl)propan-1-ol (MAJANTOL); (4-isopropylcyclohexyl)methanol (MAYOL); octahydro-2H-chromen-2-one (BICYCLO NONALACTONE); methyl non-2-ynoate (METHYL OCTYNE CARBONATE); 1-(2-naphtalenyl)-ethanone (ORANGER CRYSTALS); 4-(tert-butyl)cyclohexanol (BUTYL CYCLOHEXANOL PARA); 5-heptyldihydrofuran-2(3H)-one (PEACH PURE); 3-methyl-5-phenylpentan-1-ol (MEFROSOL); (E)-2-ethyl-4-(2,2,3-trimethylcyclopent-3-en-1-yl)but-2-en-1-ol (RADJANOL SUPER); dec-9-en-1-al (ROSALVA); 3-((1R,2S,4R,6R)-5,5,6-trimethylbicyclo[2.2.1]heptan-2-yl)cyclohexanol (SANDELA); 2-(4-methylcyclohex-3-en-1-yl)propan-2-ol (TERPINEOL); 2-isopropyl-5-methylphenol (THYMOL CRYSTALS); 2,4-dimethylcyclohex-3-enecarbaldehyde (TRICYCLAL); almond oil sweet; amyris oil; bay oil; cedarwood oil; guaiacwood oil; sandalwood oil; cabreuva oil; cocoa absolute; cade oil; chamomile oil blue; cinnamon leaf oil; cassia oil; olibanum resinoid; fenugreek absolute; fucus oil; clove bud oil; clove leaf oil; orris resinoid; labdanum oil (DYNAMONE SB); labdanum resinoid; lavender absolute; lemongrass oil; lentisque resinoid; Litsea cubeba oil; nutmeg oil; spearmint oil; mousse de chene absolute; myrrh oil; narcisse absolute; opoponax oil; orange flower absolute; osmanthus absolute; palmarosa oil; pimento berry oil; styrax oil; tagete oil; thyme absolute white; thyme oil red; tonka beans absolute; tuberose absolute; vetiver oil; violet leaf absolute; origan oil; patchouli oil; and mixtures thereof.

2. The malodour counteracting fragrance composition according to claim 1, comprising at least one fragrance ingredient drawn from Group A, more preferably at least two fragrance ingredients drawn from Group A, and most preferably at least three fragrance ingredients drawn from Group A, wherein preferably the malodour counteracting fragrance composition comprises at least one fragrance ingredient selected from the group consisting of 3,8,8,11a-tetramethyldodecahydro-1H-3,5a-epoxynaphtho[2,1-c]oxepine (AMBERKETAL); (3aR,5aS,9aS,9bR)-3a,6,6,9a-tetramethyl-2,4,5,5a,7,8,9,9b-octahydro-1H-benzo[e][1]benzofuran (AMBROFIX, AMBROXAN); benzyl 2-hydroxybenzoate (BENZYL SALICYLATE); 1-(2,6,6-trimethyl-1-cyclohex-3-enyl)but-2-en-1-one (DAMASCONE DELTA); methyl 3-oxo-2-pentylcyclopentaneacetate (HEDIONE, CEPIONATE); and (Z)-hex-3-en-1-yl 2-hydroxybenzoate (HEXENYL-3-CIS SALICYLATE).

3. The malodour counteracting fragrance composition according to claim 1, comprising more than one fragrance ingredient drawn from Group B and/or Group C, more preferably at least three fragrance ingredients drawn from Group B and/or Group C, and most preferably at least four fragrance ingredients drawn from Group B and/or Group C, wherein preferably the malodour counteracting fragrance composition comprises at least one fragrance ingredient selected from the group consisting of 3-ethoxy-4-hydroxybenzaldehyde (ETHYL VANILLIN); (E)-2-benzylideneoctanal (HEXYL CINNAMIC ALDEHYDE); mixtures of isomers (E)- and (Z)-3-methyl-4-(2,6,6-trimethyl-2-cyclohexen-1-yl)-3-buten-2-one, and (E)-1-(2,6,6-trimethylcyclohex-2-en-1-yl)pent-1-en-3-one (ISORALDEINE 70, ISORALDEINE 95); lemon oil; 4-(4-hydroxyphenyl)butan-2-one (RASPBERRY KETONE); and of 2-(4-methyl-1-cyclohex-3-enyl)propan-2-yl acetate (TERPINYL ACETATE).

4. The malodour counteracting fragrance composition according to claim 1, comprising at least five fragrance ingredients drawn from Group A and/or Group B and/or Group C, more preferably at least seven fragrance ingredients drawn from Group A and/or Group B and/or Group C, and most preferably at least eight fragrance ingredients drawn from Group A and/or Group B and/or Group C.

5. The malodour counteracting fragrance composition according to claim 1, comprising 0.01 to 20 wt % of fragrance ingredients drawn from Group A; and/or 5 to 60 wt % of fragrance ingredients drawn from Group B; and/or 1 to 50 wt % of fragrance ingredients drawn from Group C, wherein preferably the malodour counteracting fragrance composition comprises 1 to 15 wt % of fragrance ingredients drawn from Group A, and 10 to 40 wt % of fragrance ingredients drawn from Group B and/or Group C.

6. The malodour counteracting fragrance composition according to claim 1, less than 8 wt % of fragrance ingredients drawn from Group D, preferably less than 5 wt %, more preferably less than 3 wt %, and most preferably less than 1 wt %.

7. The malodour counteracting fragrance composition according to claim 1, wherein the concentrations of said fragrance ingredients are selected such that
5×[total concentration Group A]+4×[total concentration Group B]+3×[total concentration Group C]≥110;
more preferably such that
5×[total concentration Group A]+4×[total concentration Group B]+3×[total concentration Group C]≥120;
even more preferably such that
5×[total concentration Group A]+4×[total concentration Group B]+3×[total concentration Group C]≥150;
and most preferably such that
5×[total concentration Group A]+4×[total concentration Group B]+3×[total concentration Group C]≥200;

8. The malodour counteracting fragrance composition according to claim 1, wherein the concentrations of said fragrance ingredients are selected such that
4×[total concentration Group A]+4×[total concentration Group B]+3×[total concentration Group C]≥100;
more preferably such that
4×[total concentration Group A]+4×[total concentration Group B]+3×[total concentration Group C]≥110;
even more preferably such that
4×[total concentration Group A]+4×[total concentration Group B]+3×[total concentration Group C]≥120;
and most preferably such that
4×[total concentration Group A]+4×[total concentration Group B]+3×[total concentration Group C]≥150;

9. The malodour counteracting fragrance composition according to claim 1, wherein the concentrations of said fragrance ingredients are selected such that
3×[total concentration Group A]+4×[total concentration Group B]+3×[total concentration Group C]≥100;
more preferably such that
3×[total concentration Group A]+4×[total concentration Group B]+3×[total concentration Group C]≥110;
even more preferably such that
3×[total concentration Group A]+4×[total concentration Group B]+3×[total concentration Group C]≥120;
most preferably such that
3×[total concentration Group A]+4×[total concentration Group B]+3×[total concentration Group C]≥150;

10. A consumer product comprising the malodour counteracting fragrance composition according to claim 1, and a suitable carrier, wherein preferably the consumer product is a product for application to the human body, in particular a deodorant or antiperspirant product.

11. The consumer product according to claim 10, wherein the concentration of the malodour counteracting fragrance composition in the consumer product is from 0.1 wt % to 10 wt %, more preferably from 0.5 wt % to 5 wt %, and most preferably from 0.7 wt % to 3 wt %.

12. The consumer product according to claim 10, further comprising one or more skin care agents, soothing agents, deodorizing agent, probiotics, prebiotics, emollients, surfactants, thickening agents, sensates, polyols, humectants, polymers, silicones, enzymes, inorganic fillers, cationics, amphoterics, glycerides, esters, ethers, amino acids, polysaccharides, sugars, water or combinations thereof.

13. The consumer product according to claim 12, which includes a skin care agent comprising a hydrophilic Phaeodactylum tricornutum extract and/or an Orobanche rapum extract.

14. The consumer product according to claim 12, wherein the deodorizing agent comprises a zinc carboxylate salt, preferably a zinc neodecanoate, and/or a compound of formula (I) 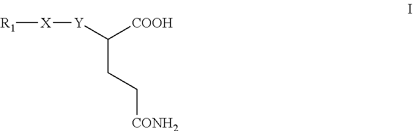
wherein Y represents a direct bond to X, or a divalent chain that may contain carbon, oxygen or nitrogen atoms, X is a zinc-chelating group, and R₁ is a linear, branched or cyclic carbon chain having about 1 to 14 carbon atoms, optionally containing one or more heteroatoms such as O, N or S, or unsaturation, the chain optionally supporting one or more substituents selected from amide, ester, keto, ether, amine, halogen or hydroxyl, or aryl or heteroaryl groups, which aryl or heteroaryl groups optionally support one or more substituents selected from amide, ester, keto, ether, amine, halogen, alkyl and/or hydroxyl.

15. The consumer product according to claim 12, comprising a prebiotic selected from the group consisting of fructans, galactans, pectin, beta-glucans, and combinations thereof.

16. A method of masking, reducing and/or preventing body malodour, said method comprising the step of topically applying the malodour counteracting fragrance composition according to an claim 1 to the human body.

17. A method of masking, reducing and/or preventing body malodour, said method comprising the step of topically applying the consumer product according to claim 10 to the human body.
